SEQUENCE LISTING (89 sequences)

SEQ ID NO: 1 - protein, 19 aa, Zea mays
MKLVLVVLAFIALVSSVSC

SEQ ID NO: 2 - protein, 19 aa, Zea mays
MKVLIVALALLALAASAAS

SEQ ID NO: 3 - protein, 19 aa, Zea mays
MKVLLVALALLALVASAAS

SEQ ID NO: 4 - protein, 19 aa, Zea mays
MRVLLVALALLALAASATS

SEQ ID NO: 5 - protein, 19 aa, Sorghum bicolor
MKVLLVALALLALAASAAS

SEQ ID NO: 6 - protein, 11 aa, Zea mays
THTSGGCGCQP

SEQ ID NO: 7 - protein, 15 aa, Sorghum bicolor
TLTTGGCGCQTPHLP

SEQ ID NO: 8 - protein, 6 aa, Zea mays
PPPVHL

SEQ ID NO: 9 - protein, 20 aa, Arabidopsis thaliana
MKTNLFLFLIFSLLLSLSSA

SEQ ID NO: 10 - protein, 6 aa, Sorghum bicolor
PPPVHL

SEQ ID NO: 11 - protein, 6 aa, Vigna unguiculata
PEPVHI

SEQ ID NO: 12 - protein, 33 aa, Zea mays
PPPPCHYPTQPPRPQPHPQPHPCPCQQPHPSPC

SEQ ID NO: 13 - protein, 41 aa, Sorghum bicolor
CHPHPTLPPHPHPCPTYPPHPSPCHPGHPGSCGVGGGPVTP

SEQ ID NO: 14 - protein, 69 aa, Artificial Sequence
  Modified protein body tag PBT-1
MKLVLVVLAFIALVSSVSCTHTSGGCGCQPPPPVHLPPPPCHYPTQPPRPQPHPQPHPCPCQQPHPSPC

SEQ ID NO: 15 - protein, 75 aa, Artificial Sequence
  Modified protein body tag PBT-2
MKLVLVVLAFIALVSSVSCTHTSGGCGCQPPPPVHLPPPVHLPPPPCHYPTQPPRPQPHPQPHPCPCQQPHPSPC

SEQ ID NO: 16 - protein, 81 aa, Artificial Sequence
  Modified protein body tag PBT-3
MKLVLVVLAFIALVSSVSCTHTSGGCGCQPPPPVHLPPPVHLPPPVHLPPPPCHYPTQPPRPQPHPQPHPCPCQQPHPSPC

SEQ ID NO: 17 - protein, 82 aa, Artificial Sequence
  Modified protein body tag PBT-4
MKTNLFLFLIFSLLLSLSSATHTSGGCGCQPPPPVHLPPPVHLPPPVHLPPPPCHYPTQPPRPQPHPQPHPCPCQQPHPSPC

SEQ ID NO: 18 - protein, 111 aa, Artificial Sequence
  Modified protein body tag PBT-5
MKVLIVALALLALAASAASTHTSGGCGCQFRGRVHIAEPVHIPEPVHIPEPVHIPEPVHIPEPVHIPEPVHIPEPVHIPEPGCHYPTQPPRPQPHPQPHPCPCQQPHPSPC

SEQ ID NO: 19 - protein, 111 aa, Artificial Sequence
  Modified protein body tag PBT-6
MKVLLVALALLALVASAASTHTSGGCGCQFRGRVHIAEPVHIPEPVHIPEPVHIPEPVHIPEPVHIPEPVHIPEPVHIPEPGCHYPTQPPRPQPHPQPHPCPCQQPHPSPC

SEQ ID NO: 20 - protein, 111 aa, Artificial Sequence
  Modified protein body tag PBT-7
MKLVLVVLAFIALVSSVSCTHTSGGCGCQFRGRVHIAEPVHIPEPVHIPEPVHIPEPVHIPEPVHIPEPVHIPEPVHIPEPGCHYPTQPPRPQPHPQPHPCPCQQPHPSPC

SEQ ID NO: 21 - protein, 111 aa, Artificial Sequence
  Modified protein body tag PBT-8
MRVLLVALALLALAASATSTHTSGGCGCQFRGRVHIAEPVHIPEPVHIPEPVHIPEPVHIPEPVHIPEPVHIPEPVHIPEPGCHYPTQPPRPQPHPQPHPCPCQQPHPSPC

SEQ ID NO: 22 - protein, 98 aa, Artificial Sequence
  Modified protein body tag PBT-9
MKVLLVALALLALAASAASTLTTGGCGCQTPHLPPPVHLPPPVHLPPPVHVPPPPPQCHPHPTLPPHPHPCPTYPPHPSPCHPGHPGSCGVGGGPVTP

SEQ ID NO: 23 - protein, 92 aa, Artificial Sequence
  Modified protein body tag PBT-10
MKVLLVALALLALAASAASTLTTGGCGCQTPHLPPPVHLPPPVHVPPPPPQCHPHPTLPPHPHPCPTYPPHPSPCHPGHPGSCGVGGGPVTP

SEQ ID NO: 24 - protein, 92 aa, Artificial Sequence
  Modified protein body tag PBT-11
MKVLIVALALLALAASAASTLTTGGCGCQTPHLPPPVHLPPPVHVPPPPPQCHPHPTLPPHPHPCPTYPPHPSPCHPGHPGSCGVGGGPVTP

SEQ ID NO: 25 - protein, 92 aa, Artificial Sequence
  Modified protein body tag PBT-12
MKLVLVVLAFIALVSSVSCTLTTGGCGCQTPHLPPPVHLPPPVHVPPPPPQCHPHPTLPPHPHPCPTYPPHPSPCHPGHPGSCGVGGGPVTP

SEQ ID NO: 26 - protein, 92 aa, Artificial Sequence
  Modified protein body tag PBT-13
MRVLLVALALLALAASATSTLTTGGCGCQTPHLPPPVHLPPPVHVPPPPPQCHPHPTLPPHPHPCPTYPPHPSPCHPGHPGSCGVGGGPVTP

SEQ ID NO: 27 - protein, 98 aa, Artificial Sequence
  Modified protein body tag PBT-14
MKVLIVALALLALAASAASTLTTGGCGCQTPHLPPPVHLPPPVHLPPPVHVPPPPPQCHPHPTLPPHPHPCPTYPPHPSPCHPGHPGSCGVGGGPVTP

SEQ ID NO: 28 - protein, 98 aa, Artificial Sequence
  Modified protein body tag PBT-15
MKLVLVVLAFIALVSSVSCTLTTGGCGCQTPHLPPPVHLPPPVHLPPPVHVPPPPPQCHPHPTLPPHPHPCPTYPPHPSPCHPGHPGSCGVGGGPVTP

SEQ ID NO: 29 - protein, 98 aa, Artificial Sequence
  Modified protein body tag PBT-16
MRVLLVALALLALAASATSTLTTGGCGCQTPHLPPPVHLPPPVHLPPPVHVPPPPPQCHPHPTLPPHPHPCPTYPPHPSPCHPGHPGSCGVGGGPVTP

SEQ ID NO: 30 - protein, 122 aa, Artificial Sequence
  Modified protein body tag PBT-17
MKVLIVALALLALAASAASTLTTGGCGCQTPHLPEPVHIPEPVHIPEPVHIPEPVHIPEPVHIPEPVHIPPPVHVPPPPPQCHPHPTLPPHPHPCPTYPPHPSPCHPGHPGSCGVGGGPVTP

SEQ ID NO: 31 - protein, 122 aa, Artificial Sequence
  Modified protein body tag PBT-18
MKVLLVALALLALVASAASTLTTGGCGCQTPHLPEPVHIPEPVHIPEPVHIPEPVHIPEPVHIPEPVHIPPPVHVPPPPPQCHPHPTLPPHPHPCPTYPPHPSPCHPGHPGSCGVGGGPVTP

SEQ ID NO: 32 - protein, 122 aa, Artificial Sequence
  Modified protein body tag PBT-19
MKLVLVVLAFIALVSSVSCTLTTGGCGCQTPHLPEPVHIPEPVHIPEPVHIPEPVHIPEPVHIPEPVHIPPPVHVPPPPPQCHPHPTLPPHPHPCPTYPPHPSPCHPGHPGSCGVGGGPVTP

SEQ ID NO: 33 - protein, 122 aa, Artificial Sequence
  Modified protein body tag PBT-20
MRVLLVALALLALAASATSTLTTGGCGCQTPHLPEPVHIPEPVHIPEPVHIPEPVHIPEPVHIPEPVHIPPPVHVPPPPPQCHPHPTLPPHPHPCPTYPPHPSPCHPGHPGSCGVGGGPVTP

SEQ ID NO: 34 - protein, 89 aa, Artificial Sequence
  Modified protein body tag PBT-21
MKLVLVVLAFIALVSSVSCTHTSGGCGCQPPPPVHLPPPVHLPPPVHLCHPHPTLPPHPHPCPTYPPHPSPCHPGHPGSCGVGGGPVTP

SEQ ID NO: 35 - protein, 89 aa, Artificial Sequence
  Modified protein body tag PBT-22
MKVLIVALALLALAASAASTHTSGGCGCQPPPPVHLPPPVHLPPPVHLCHPHPTLPPHPHPCPTYPPHPSPCHPGHPGSCGVGGGPVTP

SEQ ID NO: 36 - protein, 89 aa, Artificial Sequence
  Modified protein body tag PBT-23
MKVLLVALALLALVASAASTHTSGGCGCQPPPPVHLPPPVHLPPPVHLCHPHPTLPPHPHPCPTYPPHPSPCHPGHPGSCGVGGGPVTP

SEQ ID NO: 37 - protein, 111 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPC

SEQ ID NO: 38 - protein, 223 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQLQGTCGVGSTPILGQCVEFLRHQCSPTATPYCSPQCQSLRQQCCQQLRQVEPQHRYQAIFGLVLQSILQQQPQSGQVAGLLAAQIAQQLTAMCGLQQPTPCPYAAAGGVPH

SEQ ID NO: 39 - protein, 212 aa, Sorghum bicolor
MKVLLVALALLALAASAASTLTTGGCGCQTPHLPPPPVHLPPPVHLPPPVHLPPPVHVPPPPPQCHPHPTLPPHPHPCPTYPPHPSPCHPGHPGSCGVGGGPVTPPILGQCIEFLRHQCSPAATPYCSPQCQALRQQCCQQLRQVEPLHRYQAIFGVVLQSIQQQQPQGQSSPLPALMAAQIAQQLTAMCGLGVGQPSPCASCSPFAGGVHY

SEQ ID NO: 40 - protein, 295 aa, Zea mays
MKLVLVVLAFIALVSSVSCTQTGGCSCGQQQSHEQQHHPQQHHPQKQQHQPPPQHHQQQQHQQQQVHMQPQKHQQQQEVHVQQQQQQPQHQQQQQQQQHQQQHQCEGQQQHHQQSQGHVQQHEQSHEQHQGQSHEQQHQQQFQGHDKQQQPQQPQQYQQGQEKSQQQQCHCQEQQQTTRCSYNYYSSSSNLKNCHEFLRQQCSPLVMPFLQSRLIQPSSCQVLQQQCCHDLRQIEPQYIHQAIYNMVQSIIQEEQQQQPCELCGSQQATPKCGGNLDSSTIPTINVRLVPLILPK

SEQ ID NO: 41 - protein, 183 aa, Zea mays
MKVLIVALALLALAASAASSTSGGCGCQTPPFHLPPPFYMPPPFYLPPQQQPQPWQYPTQPPQLSPCQQFGSCGVGSVGSPFLGQCVEFLRHQCSPAATPYGSPQCQALQQQCCHQIRQVEPLHRYQATYGVVLQSFLQQQPQGELAALMAAQVAQQLTAMCGLQLQQPGPCPCNAAAGGVYY

SEQ ID NO: 42 - protein, 178 aa, Zea mays
MKVLLVALALLALVASAASTTSGGCGCQTPPFHLPPPFYLPPPVYEPPPQQPQPWQYPTQPPQLSPCQQLGSCGVGSVVSPFLGQCVEFLRHQCSPAATPYGSPQCQALQQQCCHQIRRGPAVLPAAAAAGRARGADGGPGSAAADGDVRSAAAAAARSLPLQRSCRRCLLLRKLWVL

SEQ ID NO: 43 - protein, 56 aa, Vigna unguiculata
FRGRVHIAEPVHIPEPVHIPEPVHIPEPVHIPEPVHIPEPVHIPEPVHIPEPGLGD

SEQ ID NO: 44 - protein, 304 aa, Artificial Sequence
  Consensus sequence
MKVLLVALALLALAASAXSTXTXGGCGCQXPXXXXXXXXXXXXXXXXXXHLPPPXVHLPPPVHLPPPVHLPPPVHXPPPXXXXXXXXXXPPPQXXPXPXLPPXPHHCPTQPPXXQXXXXXXXXXXXXXXXXXXXXXXXXXXXXXQXHPXPCQPXHPXXCQXGGXXXXXXXXXXXXXXXXXXXXXXGXXSXPILGQCXEFLRHQCSPXATPYCSPXXXXXXQCQXLRQQCCQQLRQVEPQHRYQAIFGXVLQSIQQQQPQXXSXXXXXLXAAQIAQQLTAMCGLXXXXPXPXAXXXXXXXXXXXX

SEQ ID NO: 45 - protein, 89 aa, Artificial Sequence
  Modified protein body tag PBT-24
MRVLLVALALLALAASATSTHTSGGCGCQPPPPVHLPPPVHLPPPVHLCHPHPTLPPHPHPCPTYPPHPSPCHPGHPGSCGVGGGPVTP

SEQ ID NO: 46 - protein, 85 aa, Artificial Sequence
  Modified protein body tag PBT-25
MKLVLVVLAFIALVSSVSCTLTTGGCGCQTPHLPPPPVHLPPPVHLPPPVHLPPPPCHYPTQPPRPQPHPQPHPCPCQQPHPSPC

SEQ ID NO: 47 - protein, 83 aa, Artificial Sequence
  Modified protein body tag PBT-26
MKLVLVVLAFIALVSSVSCTHTSGGCGCQPPPPVHLPPPVHLCHPHPTLPPHPHPCPTYPPHPSPCHPGHPGSCGVGGGPVTP

SEQ ID NO: 48 - protein, 83 aa, Artificial Sequence
  Modified protein body tag PBT-27
MKVLIVALALLALAASAASTHTSGGCGCQPPPPVHLPPPVHLCHPHPTLPPHPHPCPTYPPHPSPCHPGHPGSCGVGGGPVTP

SEQ ID NO: 49 - protein, 83 aa, Artificial Sequence
  Modified protein body tag PBT-28
MKVLLVALALLALVASAASTHTSGGCGCQPPPPVHLPPPVHLCHPHPTLPPHPHPCPTYPPHPSPCHPGHPGSCGVGGGPVTP

SEQ ID NO: 50 - protein, 83 aa, Artificial Sequence
  Modified protein body tag PBT-29
MRVLLVALALLALAASATSTHTSGGCGCQPPPPVHLPPPVHLCHPHPTLPPHPHPCPTYPPHPSPCHPGHPGSCGVGGGPVTP

SEQ ID NO: 51 - protein, 79 aa, Artificial Sequence
  Modified protein body tag PBT-30
MKLVLVVLAFIALVSSVSCTLTTGGCGCQTPHLPPPPVHLPPPVHLPPPPCHYPTQPPRPQPHPQPHPCPCQQPHPSPC

SEQ ID NO: 52 - protein, 106 aa, Artificial Sequence
  Modified protein body tag PBT-31
MKLVLVVLAFIALVSSVSCTHTSGGCGCQPEPVHIPEPVHIPEPVHIPEPVHIPEPVHIPEPVHICHPHPTLPPHPHPCPTYPPHPSPCHPGHPGSCGVGGGPVTP

SEQ ID NO: 53 - protein, 106 aa, Artificial Sequence
  Modified protein body tag PBT-32
MKVLIVALALLALAASAASTHTSGGCGCQPEPVHIPEPVHIPEPVHIPEPVHIPEPVHIPEPVHICHPHPTLPPHPHPCPTYPPHPSPCHPGHPGSCGVGGGPVTP

SEQ ID NO: 54 - protein, 106 aa, Artificial Sequence
  Modified protein body tag PBT-33
MKVLLVALALLALVASAASTHTSGGCGCQPEPVHIPEPVHIPEPVHIPEPVHIPEPVHIPEPVHICHPHPTLPPHPHPCPTYPPHPSPCHPGHPGSCGVGGGPVTP

SEQ ID NO: 55 - protein, 106 aa, Artificial Sequence
  Modified protein body tag PBT-34
MRVLLVALALLALAASATSTHTSGGCGCQPEPVHIPEPVHIPEPVHIPEPVHIPEPVHIPEPVHICHPHPTLPPHPHPCPTYPPHPSPCHPGHPGSCGVGGGPVTP

SEQ ID NO: 56 - protein, 8 aa, Artificial Sequence
  Histidine tag
HHHHHHHH

SEQ ID NO: 57 - protein, 59 aa, Artificial Sequence
  Modified protein body tag PBT-1 without spacer
MKLVLVVLAFIALVSSVSCPPPPVHLPPPPCHYPTQPPRPQPHPQPHPCPCQQPHPSPC

SEQ ID NO: 58 - DNA, 177 bp, Artificial Sequence
  Modified protein body tag PBT-1 without spacer
atgaagcttgtcctcgttgtgctcgcgtttatcgcgcttgtcagctcagttagctgcccacctccacctgtgcatctgcctccgccgccgtgtcactacccgacccagccacctaggccccaacctcatccccagccgcacccatgcccctgccagcaaccacaccccagcccctgc

SEQ ID NO: 59 - protein, 71 aa, Artificial Sequence
  Modified protein body tag PBT-3 without spacer
MKLVLVVLAFIALVSSVSCPPPPVHLPPPVHLPPPVHLPPPPCHYPTQPPRPQPHPQPHPCPCQQPHPSPC

SEQ ID NO: 60 - DNA, 213 bp, Artificial Sequence
  Modified protein body tag PBT-3 without spacer
atgaagcttgtcctcgttgtgctcgcgtttatcgcgcttgtcagctcagttagctgcccacctccacctgtgcatctgccgccacccgtgcatctgccaccgcctgtccacctccctccgccgccgtgtcactacccgacccagccacctaggccccaacctcatccccagccgcacccatgcccctgccagcaaccacaccccagcccctgc

SEQ ID NO: 61 - protein, 101 aa, Artificial Sequence
  Modified protein body tag PBT-8 without spacer
MRVLLVALALLALAASATSFRGRVHIAEPVHIPEPVHIPEPVHIPEPVHIPEPVHIPEPVHIPEPVHIPEPGCHYPTQPPRPQPHPQPHPCPCQQPHPSPC

SEQ ID NO: 62 - DNA, 303 bp, Artificial Sequence
  Modified protein body tag PBT-8 without spacer
atgagggtccttctggttgcgctcgcgctgctggcgcttgctgcgtcagcaacttcattcaggggccgcgtgcatattgccgaaccggtccacattccagaaccagttcacatcccggagccggtccatatccctgagcctgtccatatcccggagcctgtgcacatcccagagccagtgcacatacccgagcccgttcacattcccgaacccggctgtcactacccgacccagccacctaggccccaacctcatccccagccgcacccatgcccctgccagcaaccacaccccagcccctgc

SEQ ID NO: 63 - protein, 84 aa, Artificial Sequence
  Modified protein body tag PBT-9 without spacer
MKVLLVALALLALAASAASPPPVHLPPPVHLPPPVHVPPPPPQCHPHPTLPPHPHPCPTYPPHPSPCHPGHPGSCGVGGGPVTP

SEQ ID NO: 64 - DNA, 252 bp, Artificial Sequence
  Modified protein body tag PBT-9 without spacer
atgaaggtccttctcgttgcgctcgcgctgctggcgcttgccgcaagcgcggcttcccccccgccggtgcacctccctccaccagtccatctgccacctccagtccatgtcccaccacctccgccccagtgccacccgcatcccacacttccgccgcaccctcatccgtgcccgacgtaccctccccacccgtccccgtgtcatcctggccatccgggctcttgtggcgtcggaggtgggcccgtcacccca

SEQ ID NO: 65 - DNA, 207 bp, Artificial Sequence
  Modified protein body tag PBT-1
atgaagcttgtcctcgttgtgctcgcgtttatcgcgcttgtcagctcagttagctgcacccacaccagcggtgggtgtggctgccagccacctccacctgtgcatctgcctccgccgccgtgtcactacccgacccagccacctaggccccaacctcatccccagccgcacccatgcccctgccagcaaccacaccccagcccctgc

SEQ ID NO: 66 - DNA, 243 bp, Artificial Sequence
  Modified protein body tag PBT-3
atgaagcttgtcctcgttgtgctcgcgtttatcgcgcttgtcagctcagttagctgcacccacaccagcggtgggtgtggctgccagccacctccacctgtgcatctgccgccacccgtgcatctgccaccgcctgtccacctccctccgccgccgtgtcactacccgacccagccacctaggccccaacctcatccccagccgcacccatgcccctgccagcaaccacaccccagcccctgc

SEQ ID NO: 67 - DNA, 246 bp, Artificial Sequence
  Modified protein body tag PBT-4
atgaagaccaaccttttcctcttcctcatcttttcactcctgctgtctctgtccagcgccacccacaccagcggtgggtgtggctgccagccacctccacctgtgcatctgccgccacccgtgcatctgccaccgcctgtccacctccctccgccgccgtgtcactacccgacccagccacctaggccccaacctcatccccagccgcacccatgcccctgccagcaaccacaccccagcccctgc

SEQ ID NO: 68 - DNA, 333 bp, Artificial Sequence
  Modified protein body tag PBT-6
atgaaggtccttctcgttgcgctcgcgctgctggcgcttgtcgcgtcagcagcttcaacccacaccagcggtgggtgtggctgccagttcaggggccgcgtgcatattgccgaaccggtccacattccagaaccagttcacatcccggagccggtccatatccctgagcctgtccatatcccggagcctgtgcacatcccagagccagtgcacatacccgagcccgttcacattcccgaacccggctgtcactacccgacccagccacctaggccccaacctcatccccagccgcacccatgcccctgccagcaaccacaccccagcccctgc

SEQ ID NO: 69 - DNA, 333 bp, Artificial Sequence
  Modified protein body tag PBT-8
atgagggtccttctggttgcgctcgcgctgctggcgcttgctgcgtcagcaacttcaacccacaccagcggtgggtgtggctgccagttcaggggccgcgtgcatattgccgaaccggtccacattccagaaccagttcacatcccggagccggtccatatccctgagcctgtccatatcccggagcctgtgcacatcccagagccagtgcacatacccgagcccgttcacattcccgaacccggctgtcactacccgacccagccacctaggccccaacctcatccccagccgcacccatgcccctgccagcaaccacaccccagcccctgc

SEQ ID NO: 70 - DNA, 294 bp, Artificial Sequence
  Modified protein body tag PBT-9
atgaaggtccttctcgttgcgctcgcgctgctggcgcttgccgcaagcgcggcttccacactgaccacaggtggctgtggatgccagactccccatctccccccgccggtgcacctccctccaccagtccatctgccacctccagtccatgtcccaccacctccgccccagtgccacccgcatcccacacttccgccgcaccctcatccgtgcccgacgtaccctccccacccgtccccgtgtcatcctggccatccgggctcttgtggcgtcggaggtgggcccgtcacccca

SEQ ID NO: 71 - DNA, 366 bp, Artificial Sequence
  Modified protein body tag PBT-17
atgaaggtccttattgttgcgctcgcgctgctggcgcttgctgcgtcagcagcttcaacactgaccacaggtggctgtggatgccagactccccatctccctgagcctgtgcatatcccagagccagtgcacatccctgagcctgtgcacattccagagccagtgcatattccggagcccgtccatatacccgaaccggtgcatataccacctccagtccatgtcccaccacctccgccccagtgccacccgcatcccacacttccgccgcaccctcatccgtgcccgacgtaccctccccacccgtccccgtgtcatcctggccatccgggctcttgtggcgtcggaggtgggcccgtcacccca

SEQ ID NO: 72 - DNA, 366 bp, Artificial Sequence; identical to SEQ ID NO: 71
  Modified protein body tag PBT-18

SEQ ID NO: 73 - DNA, 366 bp, Artificial Sequence
  Modified protein body tag PBT-19
atgaagcttgtcctcgttgtgctcgcgtttatcgcgcttgtcagctcagttagctgcacactgaccacaggtggctgtggatgccagactccccatctccctgagcctgtgcatatcccagagccagtgcacatccctgagcctgtgcacattccagagccagtgcatattccggagcccgtccatatacccgaaccggtgcatataccacctccagtccatgtcccaccacctccgccccagtgccacccgcatcccacacttccgccgcaccctcatccgtgcccgacgtaccctccccacccgtccccgtgtcatcctggccatccgggctcttgtggcgtcggaggtgggcccgtcacccca

SEQ ID NO: 74 - DNA, 366 bp, Artificial Sequence
  Modified protein body tag PBT-20
atgagggtccttctggttgcgctcgcgctgctggcgcttgctgcgtcagcaacttcaacactgaccacaggtggctgtggatgccagactccccatctccctgagcctgtgcatatcccagagccagtgcacatccctgagcctgtgcacattccagagccagtgcatattccggagcccgtccatatacccgaaccggtgcatataccacctccagtccatgtcccaccacctccgccccagtgccacccgcatcccacacttccgccgcaccctcatccgtgcccgacgtaccctccccacccgtccccgtgtcatcctggccatccgggctcttgtggcgtcggaggtgggcccgtcacccca

SEQ ID NO: 75 - DNA, 267 bp, Artificial Sequence
  Modified protein body tag PBT-21
atgaagctggtgcttgttgtgctggccttcatcgctcttgtgagctctgtgagctgcacccatacttccggcggctgcggctgccagccaccaccaccggtgcacctgccgccgccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 76 - DNA, 267 bp, Artificial Sequence
  Modified protein body tag PBT-22
atgaaggttctgatcgttgctctggccctgctggctcttgctgcttctgctgccagcacccatacttccggcggctgcggctgccagccaccaccaccggtgcacctgccgccgccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 77 - DNA, 267 bp, Artificial Sequence
  Modified protein body tag PBT-23
atgaaggttctgcttgttgctctggccctgctggctcttgtggcttctgctgccagcacccatacttccggcggctgcggctgccagccaccaccaccggtgcacctgccgccgccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 78 - DNA, 267 bp, Artificial Sequence
  Modified protein body tag PBT-24
atgagggttctgcttgttgctctggccctgctggctcttgctgcttctgctaccagcacccatacttccggcggctgcggctgccagccaccaccaccggtgcacctgccgccgccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 79 - DNA, 255 bp, Artificial Sequence
  Modified protein body tag PBT-25
atgaagctggtgctggtcgtgctcgccttcattgctctggtgtctagcgtgagctgcaccctgaccaccggcggctgcggctgccagaccccacacctcccaccacctccagttcatcttccgccaccagtgcatctgccaccacctgttcacttgccaccacctccatgccactacccaactcagccaccaaggccacagccacatcctcaaccacatccatgcccatgccagcagccacacccaagcccatgt

SEQ ID NO: 80 - DNA, 249 bp, Artificial Sequence
  Modified protein body tag PBT-26
atgaagctggtgcttgttgtgctggccttcatcgctcttgtgagctctgtgagctgcacccatacttccggcggctgcggctgccagccaccaccaccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 81 - DNA, 249 bp, Artificial Sequence
  Modified protein body tag PBT-27
atgaaggttctgatcgttgctctggccctgctggctcttgctgcttctgctgccagcacccatacttccggcggctgcggctgccagccaccaccaccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 82 - DNA, 249 bp, Artificial Sequence
  Modified protein body tag PBT-28
atgaaggttctgcttgttgctctggccctgctggctcttgtggcttctgctgccagcacccatacttccggcggctgcggctgccagccaccaccaccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 83 - DNA, 249 bp, Artificial Sequence
  Modified protein body tag PBT-29
atgagggttctgcttgttgctctggccctgctggctcttgctgcttctgctaccagcacccatacttccggcggctgcggctgccagccaccaccaccagtgcacctcccaccaccagttcatctgtgccacccacatccaaccctgccaccacatccacatccgtgcccaacctacccaccacatccttcaccatgccacccaggccaccctggttcttgcggcgttggcggcggtccagtgactcca

SEQ ID NO: 84 - DNA, 237 bp, Artificial Sequence
  Modified protein body tag PBT-30
atgaagctggtgctggtcgtgctcgccttcattgctctggtgtctagcgtgagctgcaccctgaccaccggcggctgcggctgccagaccccacacctcccaccacctccagtgcatctgccaccacctgttcacttgccaccacctccatgccactacccaactcagccaccaaggccacagccacatcctcaaccacatccatgcccatgccagcagccacacccaagcccatgt

SEQ ID NO: 85 - DNA, 318 bp, Artificial Sequence
  Modified protein body tag PBT-31
atgaagctggtgcttgttgtgctggccttcatcgctcttgtgagctctgtgagctgcacccatacttccggcggctgcggctgccagccagaaccagttcatatcccagagccggtgcacatccctgaacctgtgcatattcccgagcccgtccacattccagagccagtgcacatcccagagccagttcatatctgccacccacatccaaccctgccaccacatccacatccgtgcccaacctacccaccacatccttcaccatgccacccaggccaccctggttcttgcggcgttggcggcggtccagtgactcca

SEQ ID NO: 86 - DNA, 318 bp, Artificial Sequence
  Modified protein body tag PBT-32
atgaaggttctgatcgttgctctggccctgctggctcttgctgcttctgctgccagcacccatacttccggcggctgcggctgccagccagaaccagttcatatcccagagccggtgcacatccctgaacctgtgcatattcccgagcccgtccacattccagagccagtgcacatcccagagccagttcatatctgccacccacatccaaccctgccaccacatccacatccgtgcccaacctacccaccacatccttcaccatgccacccaggccaccctggttcttgcggcgttggcggcggtccagtgactcca

SEQ ID NO: 87 - DNA, 318 bp, Artificial Sequence
  Modified protein body tag PBT-33
atgaaggttctgcttgttgctctggccctgctggctcttgtggcttctgctgccagcacccatacttccggcggctgcggctgccagccagaaccagttcatatcccagagccggtgcacatccctgaacctgtgcatattcccgagcccgtccacattccagagccagtgcacatcccagagccagttcatatctgccacccacatccaaccctgccaccacatccacatccgtgcccaacctacccaccacatccttcaccatgccacccaggccaccctggttcttgcggcgttggcggcggtccagtgactcca

SEQ ID NO: 88 - DNA, 318 bp, Artificial Sequence
  Modified protein body tag PBT-34
atgagggttctgcttgttgctctggccctgctggctcttgctgcttctgctaccagcacccatacttccggcggctgcggctgccagccagaaccagttcatatcccagagccggtgcacatccctgaacctgtgcatattcccgagcccgtccacattccagagccagtgcacatcccagagccagttcatatctgccacccacatccaaccctgccaccacatccacatccgtgcccaacctacccaccacatccttcaccatgccacccaggccaccctggttcttgcggcgttggcggcggtccagtgactcca

SEQ ID NO: 89 - DNA, 333 bp, Zea mays
  Native 27KD gamma zein domain
atgagggtgttgctcgttgccctcgctctcctggctctcgctgcgagcgccacctccacgcatacaagcggcggctgcggctgccagccaccgccgccggttcatctaccgccgccagtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgc

(19) United States
(12) Patent Application Publication
(10) Pub. No.: US 2013/0326727 A1
(43) Pub. Date: Dec. 5, 2013

(54) Modified Protein Body Tags and Production Methods Thereof

(71) Applicants: Lahoucine Achnine, Cary, NC (US); Jill Stevenson-Paulik, Cary, NC (US); Suzy M. Cocciolone, Garner, NC (US); Durba Ghoshal, Cary, NC (US); Jeffrey A. Brown, Apex, NC (US); Libby Bernal, Durham, NC (US)

(72) Inventors: Lahoucine Achnine, Cary, NC (US); Jill Stevenson-Paulik, Cary, NC (US); Suzy M. Cocciolone, Garner, NC (US); Durba Ghoshal, Cary, NC (US); Jeffrey A. Brown, Apex, NC (US); Libby Bernal, Durham, NC (US)

(21) Appl. No.: 13/992,259

(22) PCT Filed: Dec. 8, 2011
(86) PCT No.: PCT/IB11/55551

§ 371 Date: Aug. 19, 2013

Related U.S. Application Data
(60) Provisional application No. 61/421,920, filed on Dec. 10, 2010.

(51) Int. Cl. C07K 14/415; G06F 19/10

(52) U.S. Cl. CPC C07K 14/415; G06F 19/10
USPC 800/278; 530/324; 530/350; 536/23.4; 435/320.1; 435/419; 800/298; 435/414; 435/412; 435/415; 800/317.3; 800/306; 800/320.1; 800/320.2; 800/320.3; 800/320; 800/312; 800/314; 435/7.1; 435/69.7; 703/11; 426/656

(57) ABSTRACT

The present invention belongs to genetic engineering technical field and discloses modified protein body tags, a system for evaluating the efficacy of the modified polypeptides, and methods for targeting proteins to protein bodies or for forming protein bodies. The present invention also discloses modified protein body tags with reduced allergenicity and methods for making and using the modified protein body tags.

### FIELD OF THE INVENTION

The invention relates generally to methods for modifying the accumulation of a protein of interest in a transgenic organism. The methods involve the use of modified protein body tags to induce protein body targeting and/or formation.

### BACKGROUND OF THE INVENTION

Multiple studies indicate that the targeting of heterologously expressed proteins to various cellular compartments has a major impact on protein accumulation. Specifically, the deposition of heterologously expressed proteins into protein bodies provides a mechanism for protecting the protein from uncontrolled degradation by cellular machinery. Sequestration of proteins in protein bodies has the added advantage of protecting the cell from potentially toxic proteins. One potential method of targeting heterologous proteins to protein bodies is the fusion of the heterologous protein to a protein body tag. For example, protein body targeting can be driven by the proline rich region of the 27 kDa γ-zein protein, which self assembles into protein bodies and confers stability to overexpressed heterologous proteins when expressed as fusion proteins (Geli et al., 1994, Plant Cell 6: 1911-1922; Torrent et al., 2009, BMC Biology 7: 1-14).

The maize zein proteins are part of a large family of seed storage proteins found in several plant species designated as prolamins. Prolamins have variable structures, but they share the common property of being soluble in aqueous alcohol. This characteristic distinguishes them from other seed storage proteins such as albumins (which are soluble in water), and globulins (which are soluble in dilute salt solution) (Shewry et al., 2002, J. Exp. Bot. 53: 947-958; and Holding et al., 2008, Advances in Plant Biochemistry and Molecular Biology, Vol. 1, Chapter 5, Elsevier Ltd., pp. 107-133).

Prolamins are synthesized on rough Endoplasmic Reticulum (ER) membranes and can form protein bodies in the ER or be transported into specialized protein storage vacuoles. Prolamins are typically very rich in proline and glutamine and low in lysine, tryptophan, tyrosine and threonine (Holding et al., 2008, Advances in Plant Biochemistry and Molecular Biology, Vol. 1, Chapter 5, Elsevier Ltd., pp. 107-133).

Prolamins in other species include kafirins in sorghum (Sorghum bicolor) (Belton et al., 2006, J. Cereal Science 44: 272-286), hordeins in barley (Hordeum vulgare), secalins in rye (Secale cereale), and the gliadins in wheat (Shewry et al., 1990, Biochem J. 267: 1-12). The wheat gliadins are the major components of gluten (Shewry et al., 1990, Biochem J. 267: 1-12). The wheat, barley and rye prolamins are classified into three groups based on their amino acid composition: the sulfur-rich, sulfur poor, and high molecular weight prolamins (Shewry et al., 1990, Biochem J. 267: 1-12).

In maize, the wild-type 27 kDa γ-zein protein body tag sequence (SEQ ID NO: 37) has been shown to drive protein body formation and is comprised of the first 111 amino acids of the 27 kDa γ-zein protein (SEQ ID NO: 38). The protein body tag includes four domains: the N-terminal signal peptide, a spacer region, a repeat domain comprising 7 repeats of the sequence PPPVHL (SEQ ID NO: 8), and a proline-rich domain referred to as the Pro-X domain. A depiction of these domains is shown in FIG. 1. The repeat region is inserted within other regions that are rich in cysteine residues. These cysteine residues form disulfide bonds that likely contribute to protein body assembly (Pompa et al., 2006, Plant Cell 18: 2608-2621).

The targeting of protein to Endoplasmic Reticulum (ER)-derived protein bodies via the γ-zein protein body tag has been reported to enhance protein accumulation 10-100-fold over other targeting approaches (Torrent et al., 2009, BMC Biology 7: 1-14). Because the protein body offers increased stability of the expressed protein, this approach allows for over-expression of non-storage proteins. Torrent et al. (2009) disclosed the use of γ-zein protein body tag fusions to drive protein body formation and accumulation of signal transduction proteins in tobacco leaves, insect cells, and mammalian cell cultures. Their analysis indicates that the proteins accumulate to significantly greater levels when targeted in this manner, and that the protein accumulation and protein bodies do not disturb normal cell growth and viability. A system to accumulate recombinant calcitonin in protein bodies in tobacco comprising fusing the calcitonin coding region to the N-terminus of the 27 kDa γ-zein protein has also been described (U.S. Pat. No. 7,575,898).

There remains a need to develop protein body tags that may improve protein body targeting and/or formation and accumulation of proteins of interest in various crop plants. There also remains a need for a system to evaluate the efficacy of the modified protein body tags.

Further, despite the potential advantages of γ-zein fusion proteins, the use of the wild-type 27 kDa γ-zein protein body tag sequence for ectopic overexpression in commercial cultivars may not be feasible due to its potential allergenicity. A homology search within the AllergenOnline database reveals that γ-zein domains have significant homology to known allergens. Furthermore, Krishnan et al. demonstrated that young pigs consuming maize produced antibodies to the 27 kDa γ-zein protein, and identified the protein as being a potential allergen (Krishnan et al., 2010, J. Agric. Food Chem. 58: 7323-7328).

Therefore, overexpression of the wild-type 27 kDa γ-zein protein body tag sequence in transgenic maize for human or animal consumption may be undesirable. Under present government regulations, the potential allergenicity of the 27 kDa γ-zein protein could also block regulatory approval of transgenic crops overexpressing this polypeptide. For example, if an allergenic potential of a protein is indicated in a genetically-modified crop, the Food and Drug Administration (FDA) under present government regulations requires labeling to inform consumers of the allergenic potential and may take legal action against commercialization (Kaeppler, 2000, Agron. J. 92: 793-797).

Therefore, a need also exists to develop polypeptides that are capable of directing heterologous proteins to protein bodies, but that do not raise allergenicity concerns, and/or have reduced allergenicity.

### SUMMARY OF THE INVENTION

The present invention provides modified protein body tags, a system for evaluating the efficacy of the modified polypeptides, and methods for targeting proteins to protein bodies or formation of protein bodies and accumulation of proteins of interest. Modified protein body tags which are free of identifiable homology to allergens, or of reduced homology to allergens, have also been developed.

In one embodiment, the invention provides a modified protein body tag comprising a signal peptide domain, a spacer domain, a repeat domain comprising one or more repeat units, and a Pro-X domain,

wherein
(i) at least one repeat unit of the repeat domain is heterologous to the Pro-X domain,
(ii) the signal peptide domain is from a different protein from the same species as the Pro-X domain,
(iii) at least one of the domains but not all of said domains is from a γ-kafirin protein, and/or
(iv) the spacer domain is heterologous to the repeat domain or the Pro-X domain.

In another embodiment, at least one of the domains of the modified protein body tag is obtained from a γ-zein protein or homolog thereof. In a further embodiment, the γ-zein protein or homolog thereof is selected from the group consisting of a 27 kDa γ-zein protein, a 50 kDa γ-zein protein, a 16 kDa γ-zein protein, a γ-kafirin, and a cowpea γ-zein ortholog.

In a further embodiment, the invention provides a modified protein body tag comprising a signal peptide domain, a spacer domain, a repeat domain comprising one or more repeat units, and a Pro-X domain, wherein at least one domain is from a γ-kafirin protein and the repeat domain has a different number of repeats units than a wild-type γ-kafirin repeat domain.

In one aspect of the invention, the modified protein body tag comprises one or more domain comprising the polypeptide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and/or SEQ ID NO: 13, or functional variants thereof. In another aspect, the invention provides one or more nucleic acid molecule encoding the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and/or SEQ ID NO: 13, or functional variants thereof.

In a further embodiment, the invention relates to a modified protein body tag comprising the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55, or functional variants thereof, or to nucleic acid molecules encoding the amino acid sequence.

In another embodiment, the invention also relates to nucleic acids which encode the modified protein body tags, to the complement of the nucleic acids, and to nucleic acids that hybridize to these nucleic acids. The invention also provides for expression cassettes, vectors, host cells, plants or parts thereof which comprise such nucleic acids. The invention further relates to constructs and fusion proteins which comprise one or more proteins of interest associated with the modified protein body tags, preferably as fusion proteins.

In a further embodiment, the invention also relates to nucleic acids which encode a modified protein body tag and which comprise the sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88, or a functional variant thereof.

In another embodiment, host cell systems and methods for evaluating protein body targeting and/or formation and/or accumulation of the protein of interest which utilize the modified protein body tags or nucleic acids encoding these tags are provided.

In one embodiment, the invention provides a host cell system for evaluating protein body targeting and/or formation and/or accumulation of a protein of interest in a protein body, comprising one or more host cells which comprise
a) a nucleic acid molecule comprising a nucleic acid sequence encoding a modified protein body tag of the invention; and
b) at least one nucleic acid molecule encoding a protein of interest.

In a further embodiment, the invention provides a method for evaluating protein body targeting and/or formation and/or accumulation of a protein of interest in a protein body, comprising
a) providing the host cell system of the invention; and
b) evaluating protein body formation and/or expression and/or accumulation of the protein of interest in the host cells of said system.

In another embodiment, a method for producing a modified protein body tag with reduced homology to recognized allergenic sequences relative to a corresponding wild-type protein body tag is disclosed.

In another embodiment, the invention provides a method for designing a protein body tag of reduced allergenicity relative to a corresponding wild-type protein body tag, which comprises
a) providing amino acid sequences which encode the signal peptide domain, spacer domain, repeat domain, and Pro-X domain of a protein body tag, which sequences together define the amino acid sequence of a designed protein body tag;
b) comparing the sequence of said designed protein body tag to a database of allergenic proteins to identify areas of homology, if any, between the designed protein body tag and the proteins contained in the database, which areas of homology signify potential allergenicity; and
c) identifying designed protein body tags having no or few areas of homology which signify potential allergenicity as indicated by said comparison.

In a further embodiment of the method for designing a protein body tag of reduced allergenicity, the areas of potential allergenicity are defined by 8 contiguous amino acids or are defined by 80 contiguous amino acids.

In still another aspect, the invention concerns products produced by or from the plants of the invention, their plant parts, their seeds, or their progeny, which comprise the nucleic acid molecule or expression cassette of the invention, such as a foodstuff, feedstuff, food supplement, feed supplement, fiber, cosmetics or pharmaceuticals.

The invention further provides certain polynucleotides which encode the polypeptides identified in FIG. 3, and certain polypeptides identified in FIG. 3. The invention is also embodied in recombinant vectors comprising a polynucleotide of the invention.

In yet another embodiment, the invention concerns a method of producing a transgenic plant, wherein the method comprises transforming a plant cell with an expression vector comprising a polynucleotide of the invention, and generating from the plant cell a transgenic plant that expresses the polypeptide encoded by the polynucleotide. Expression of the polypeptide in the plant results in the one or more protein of interest being targeted to protein bodies.

In still another embodiment, the invention provides a method for targeting a protein of interest to a protein body. The method comprises the steps of transforming a plant cell with an expression cassette comprising a polynucleotide encoding the polypeptide of FIG. 3 and a protein of interest to form protein bodies in said cell.

The invention further provides a method for production of a protein of interest comprising (a) culturing or growing the plant cell, plant tissue, plant or part thereof or transgenic cells, cell cultures, parts, tissues, organs or propagation material derived therefrom under conditions that provide for expression of the protein of interest; and optionally (b) isolating the desired protein of interest.

In another aspect, the invention relates to a method for the production of a foodstuff, feedstuff, seed, pharmaceutical, or protein of interest comprising (a) growing or culturing the plant cell, plant tissue, plant or part thereof or transgenic cells, cell cultures, parts, tissues, organs or propagation material derived therefrom; and (b) producing and/or isolating the desired foodstuff, feedstuff, seed, pharmaceutical, or protein of interest from the plant cell, plant tissue, plant or part thereof or transgenic cells, cell cultures, parts, tissues, organs or propagation material derived therefrom.

In yet a further embodiment, the invention provides a method of producing a transgenic plant which targets a protein of interest to a protein body, the method comprising:
a) transforming a plant cell with an expression cassette comprising
   i) a first nucleotide sequence comprising a nucleotide sequence encoding the modified protein body tag as described herein; and
   ii) a second nucleotide sequence encoding a protein of interest; and
b) regenerating a transgenic plant from the plant cell.

In a further embodiment, this expression cassette may comprise at least one other nucleotide sequence encoding a further protein of interest, which can be overexpressed or downregulated. An example of a further protein of interest is an α-zein protein.

In a further aspect of the invention, the modified protein body tags may improve protein body formation and/or improve targeting and/or accumulation of proteins to protein bodies relative to wild-type protein body tags. In a further embodiment, the invention relates to a method for improving protein body formation (e.g. number of protein bodies, or size of protein bodies) and/or improving targeting and/or accumulation of proteins to protein bodies in a transgenic plant relative to a corresponding wild-type plant comprising growing a transgenic plant cell, plant or part thereof which comprises the modified protein body tag of the invention.

### BRIEF DESCRIPTION OF THE FIGURES

FIG. 1 shows the domain structure of the 27 kDa γ-zein domain polypeptide. The regions of the protein capable of protein body self-assembly are the signal peptide, spacer, repeat domain, and Pro-X domain.

FIG. 2 shows the alignment of the protein sequences of the 50 kDa γ-zein protein (AAL16979, SEQ ID NO: 40), the 27 kDa γ-zein protein (AAL16977, SEQ ID NO: 38), the sorghum γ-kafirin (ADD98900.1, SEQ ID NO: 39), the cowpea glutelin 2 partial sequence (AAD34914 glutelin, SEQ ID NO: 43), and a consensus therebetween (SEQ ID NO: 44). The glutelin 2 sequence includes repeat units and a portion of the Pro-X domain at the N-terminus downstream of the repeat domain and a spacer between the signal peptide and the repeat domain.

FIG. 3 depicts the sequences of various modified protein body tags and sequences from Tables 2 and 8.

FIG. 4 depicts the sequences of certain wild-type seed storage proteins, a wild-type γ-zein protein body tag, and the N-terminal proline-rich domain of γ-zein called Zera (Llop-Tous et al., 2010, J. Biol. Chem. 285 (46): 35633-44). The various domains of the protein body tag are identified as follows: the Signal Peptide is in bold, the Spacer in lower case, the Repeat Domain is underlined, a Single Repeat Unit in the Repeat Domain is underlined and in italics, and the Pro-X Domain is in italics.

FIG. 5 provides an example of a construct comprising a protein body tag (PBT), a 8×His-tag, and the C-terminus of SEQ ID NO: 38 (corresponding to positions 112 to 223 of the amino acid sequence of SEQ ID NO: 38) which can be used for analysis of protein body formation.

FIG. 6 provides an example of an Immunoblot analysis of 8×His-tagged PBT fusions with the C-terminus of SEQ ID NO: 38 over-expressed in BMS maize cell cultures, and His-tagged SEQ ID NO: 38 as a control.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides modified protein body tags which can be derived from prolamins such as zein proteins. The modified protein body tags may comprise one or more domains from any prolamin or any zein protein, and the invention is not limited to specific sources of the one or more domains except as specified in the claims.

The term “zein” encompasses a family of several related maize proteins. The zeins are rich in proline, glutamine, leucine and/or alanine and can be extracted in aqueous alcohol solutions in the presence of a reducing agent. Zeins can be divided into four structurally distinct types (α, β, γ, and δ) based on differences in solubility, amino acid sequence, and electrophoretic, chromatographic, and immunological properties. The α-zeins include 21-25 kDa polypeptides and constitute 75-85% of total zeins. The β-zeins include 17-18 kDa methionine-rich polypeptides and constitute 10-15% of total zeins. The γ-zeins include a 27 kDa proline-rich polypeptide that constitutes 5-10% of total zeins (Esen, 1987, J Cereal Science 5: 117-128) as well as polypeptides of 16 kDa (AAL16978 and ABD63259) and 50 kDa (AF371263.1, Woo et al., 2001, Plant Cell 13: 2297-2317). The γ-zeins include proteins of 10 and 18 kDa (Woo et al., 2001, Plant Cell 13: 2297-2317).

Prolamin proteins from species other than maize have also been divided into structurally distinct types. For example, the kafirins from sorghum may be classified into α-kafirins (24 and 26 kDa), β-kafirins (16, 18 and 20 kDa), and γ-kafirins (28 kDa). The α-prolamin is the major storage protein of grains. After synthesis, kafirins and zeins are translocated to the lumen of the rough ER where they accumulate and are packaged into discrete protein bodies about 1 μm in diameter. Protein bodies are structured such that α-prolamins are located centrally with most of the γ-prolamin and some β-prolamin at the body periphery in sorghum.

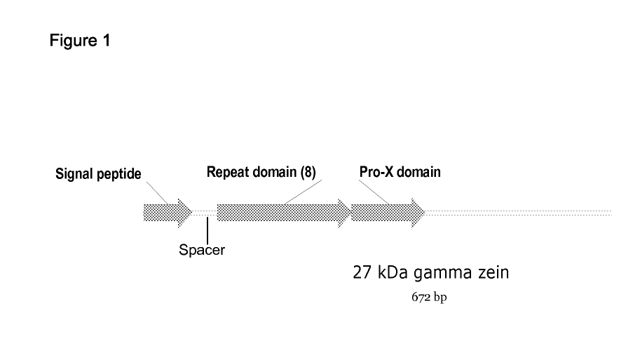

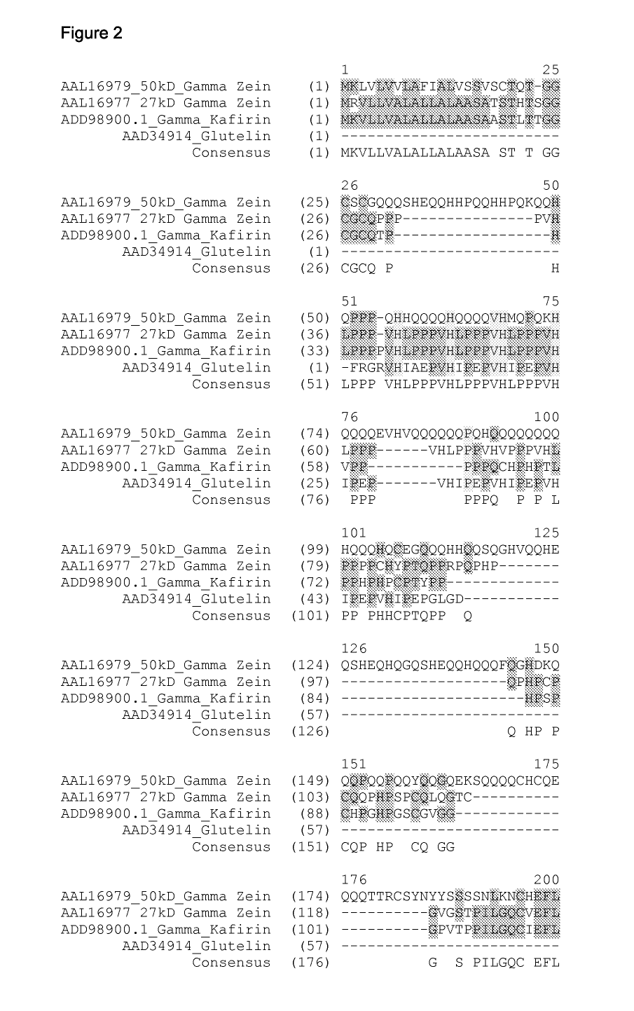

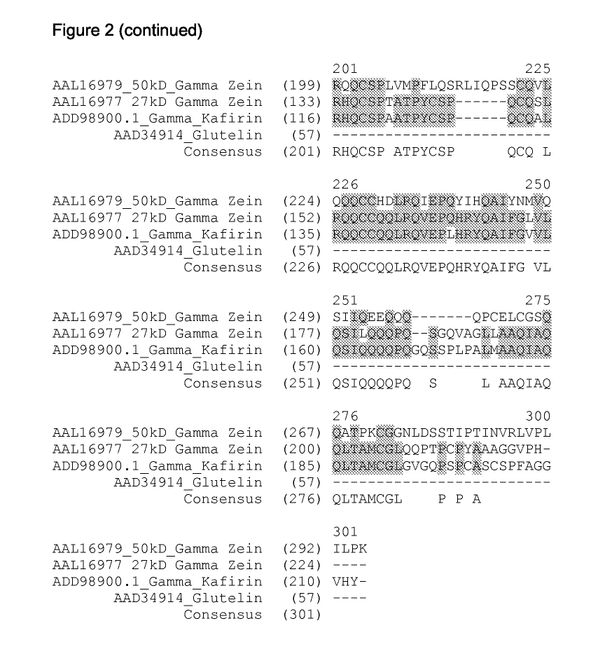

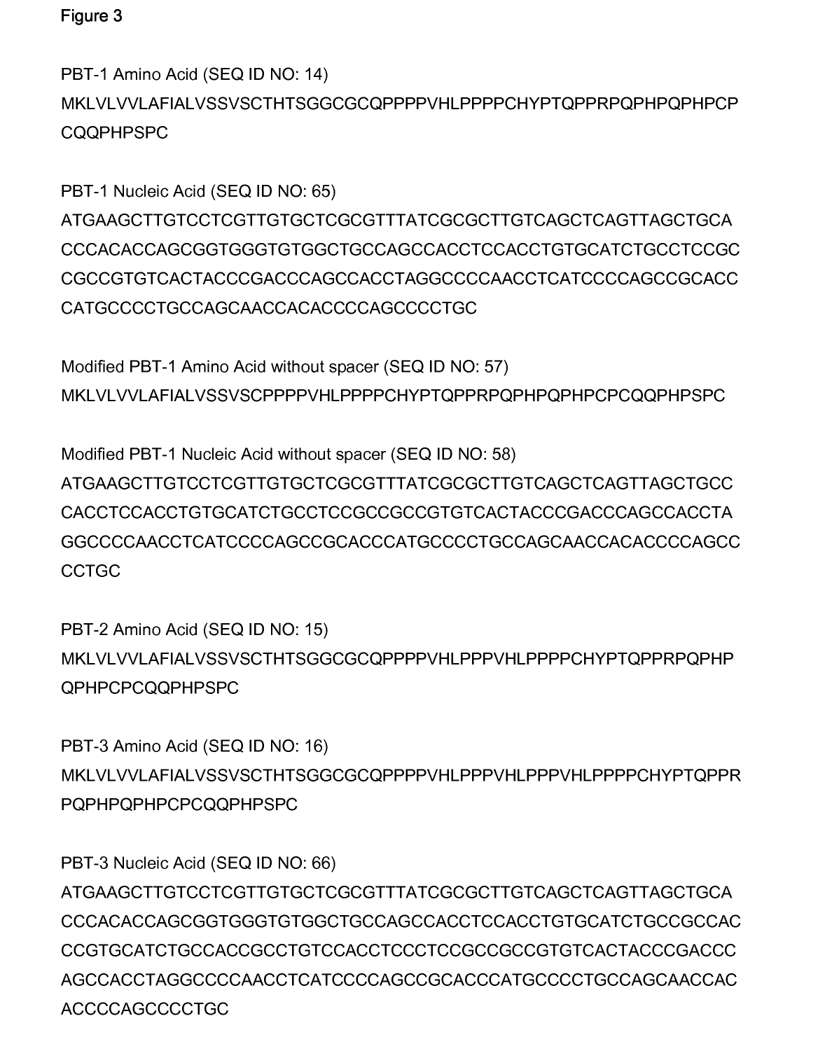

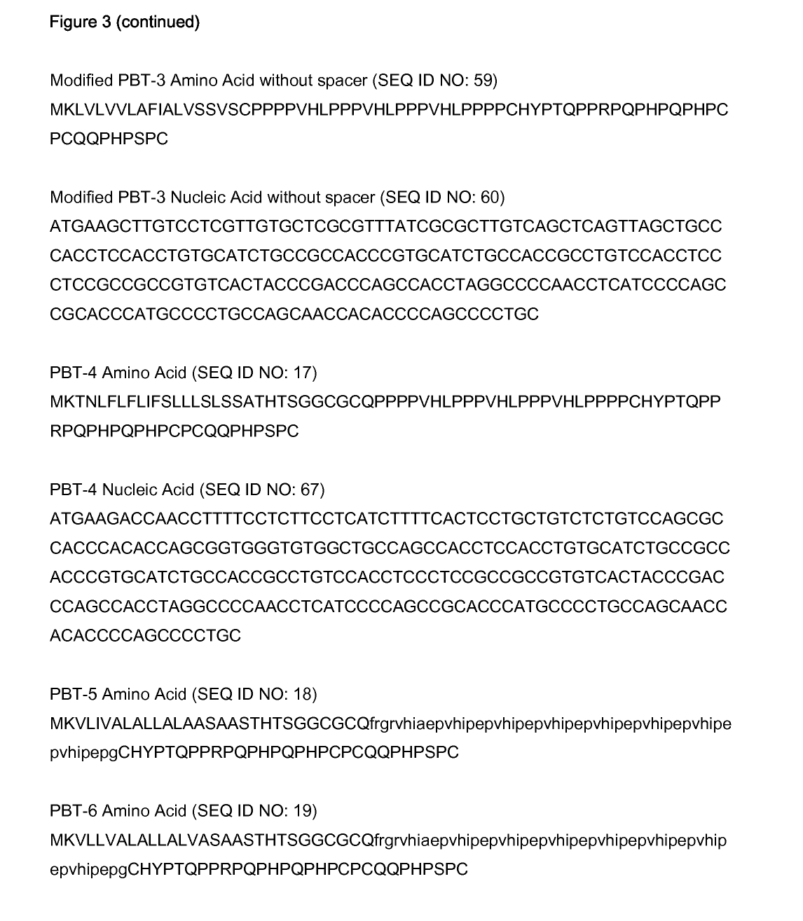

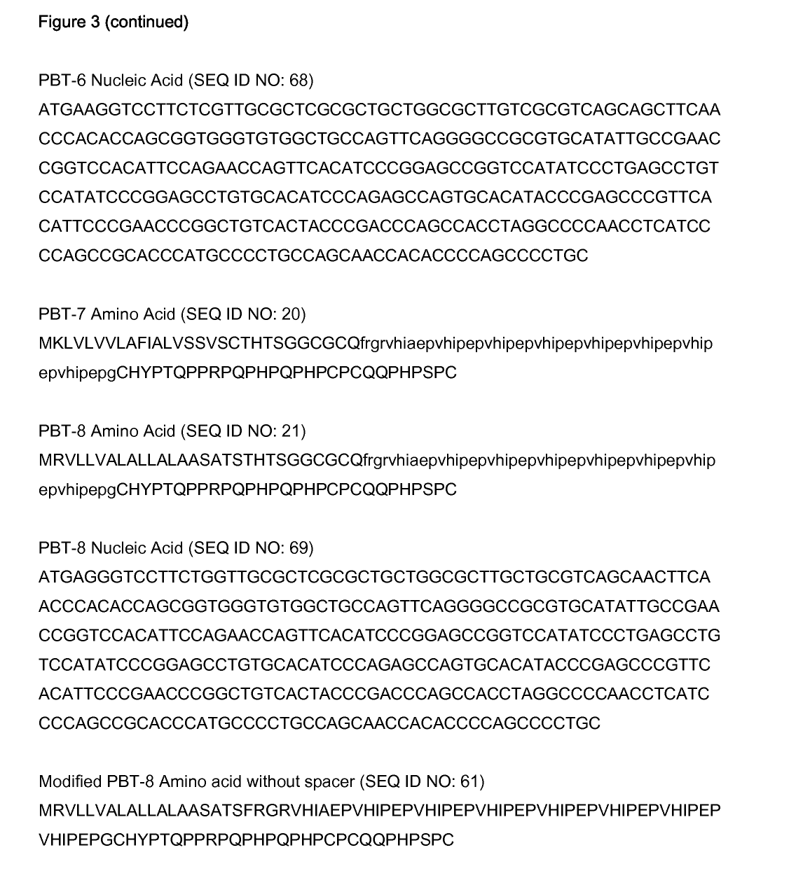

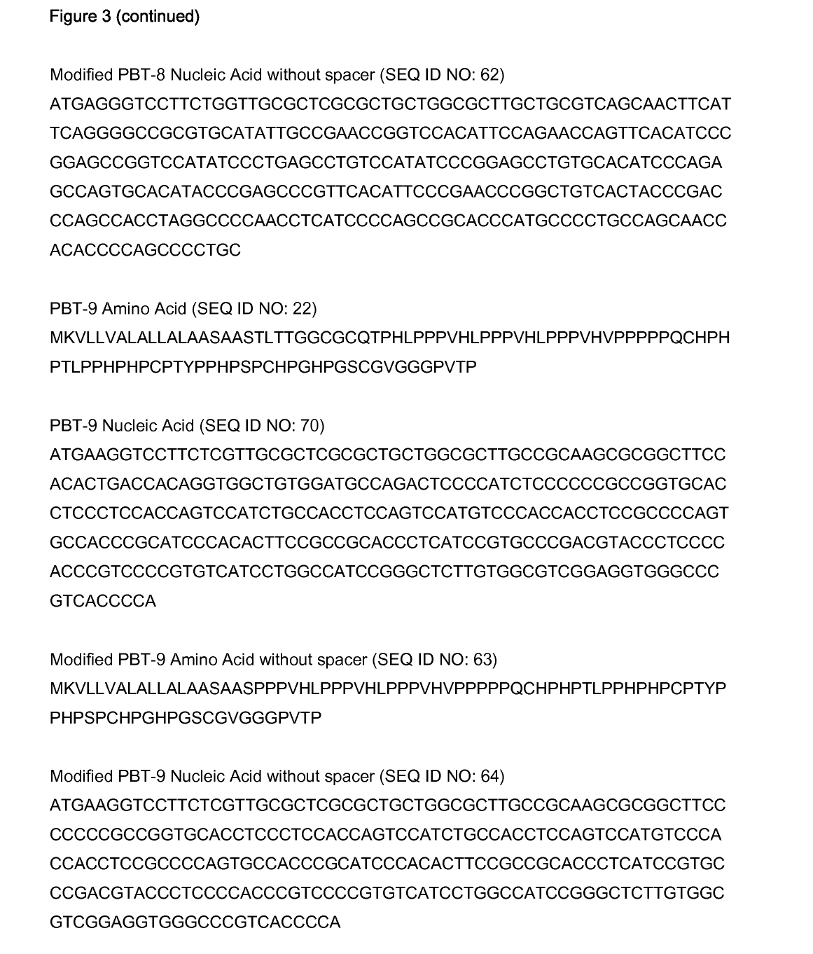

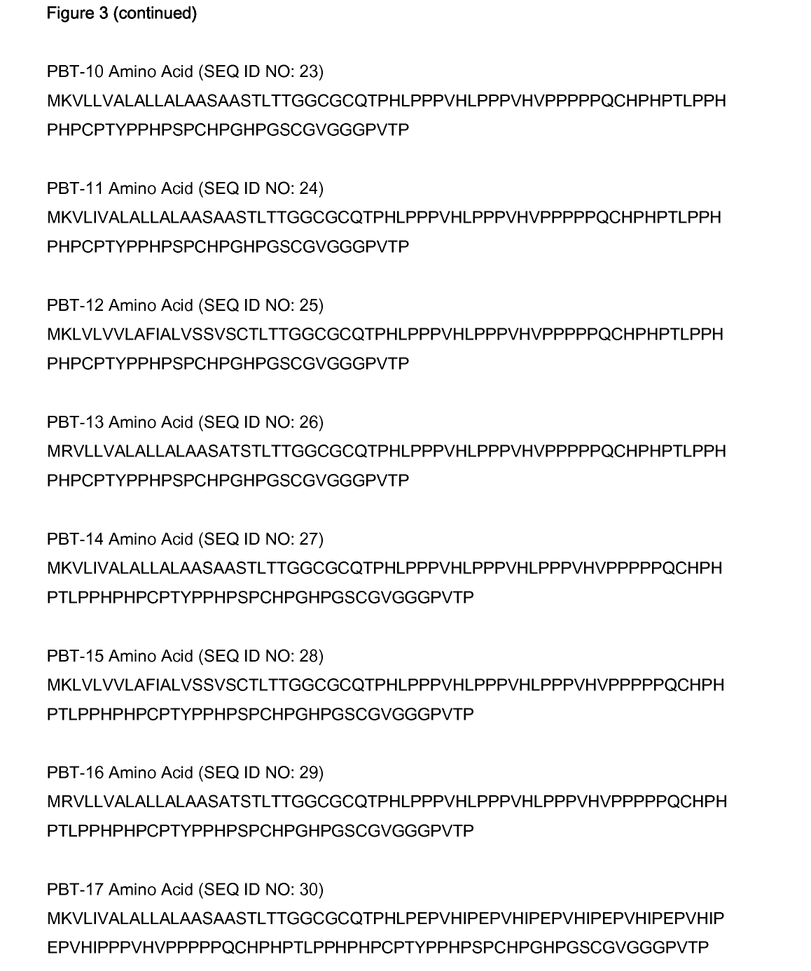

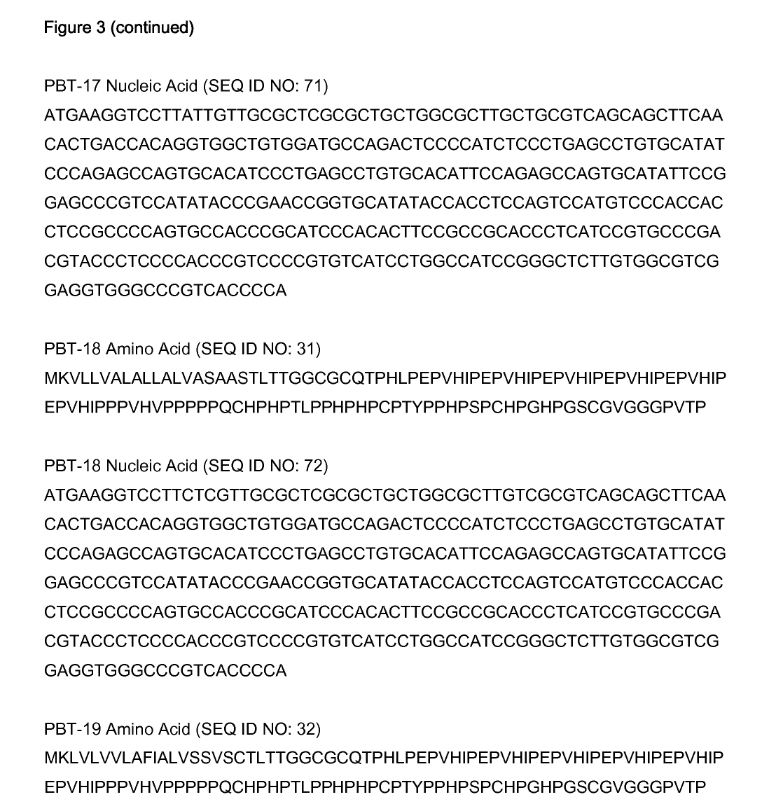

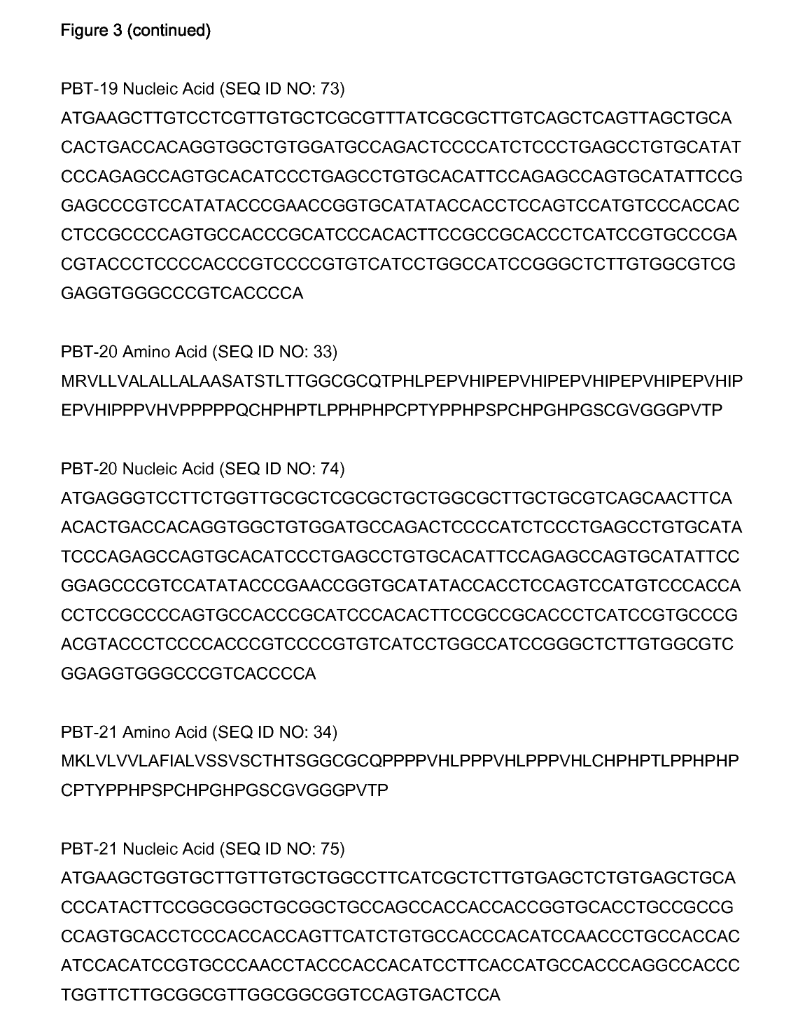

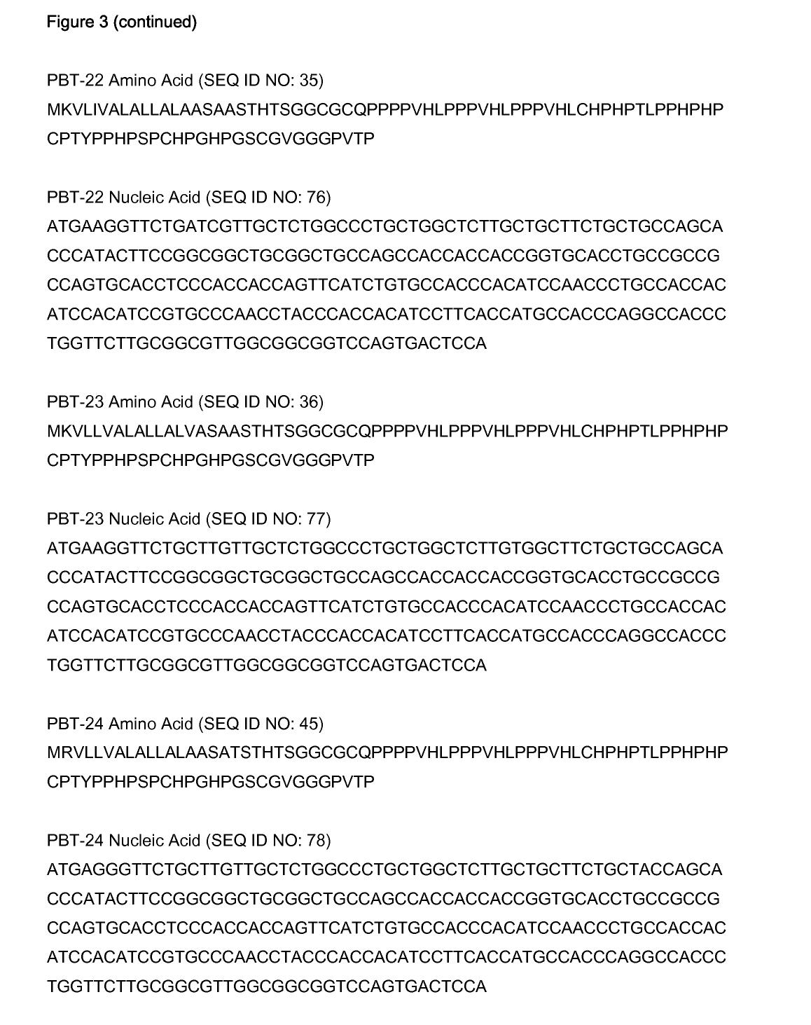

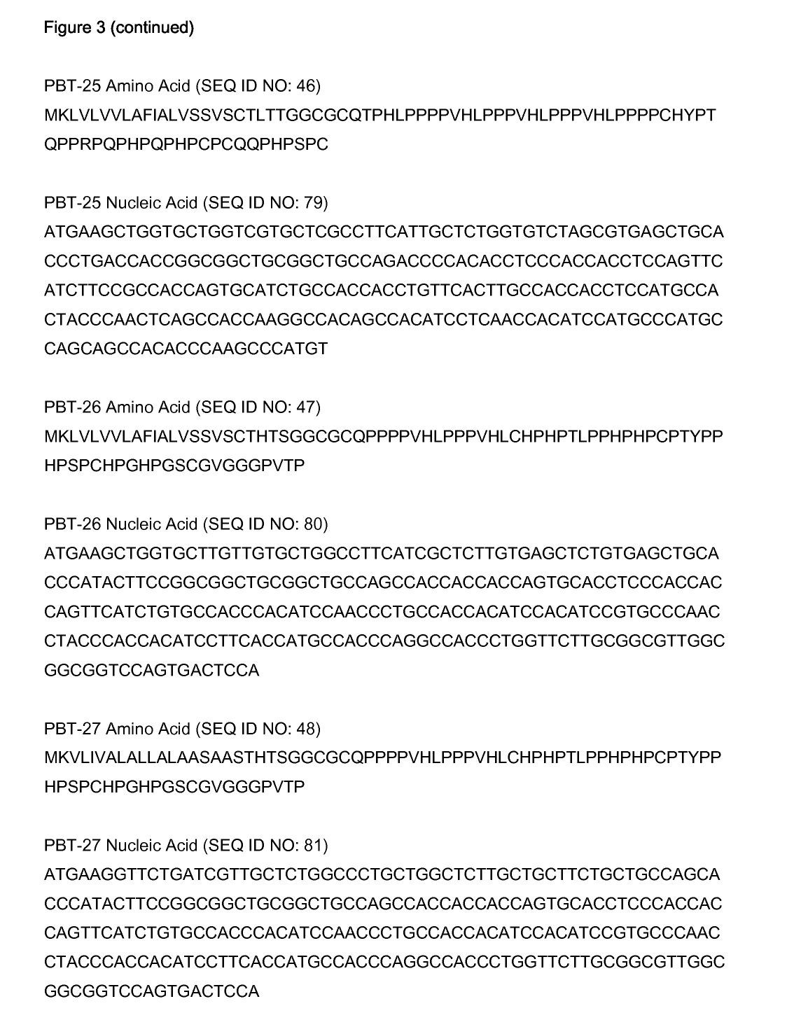

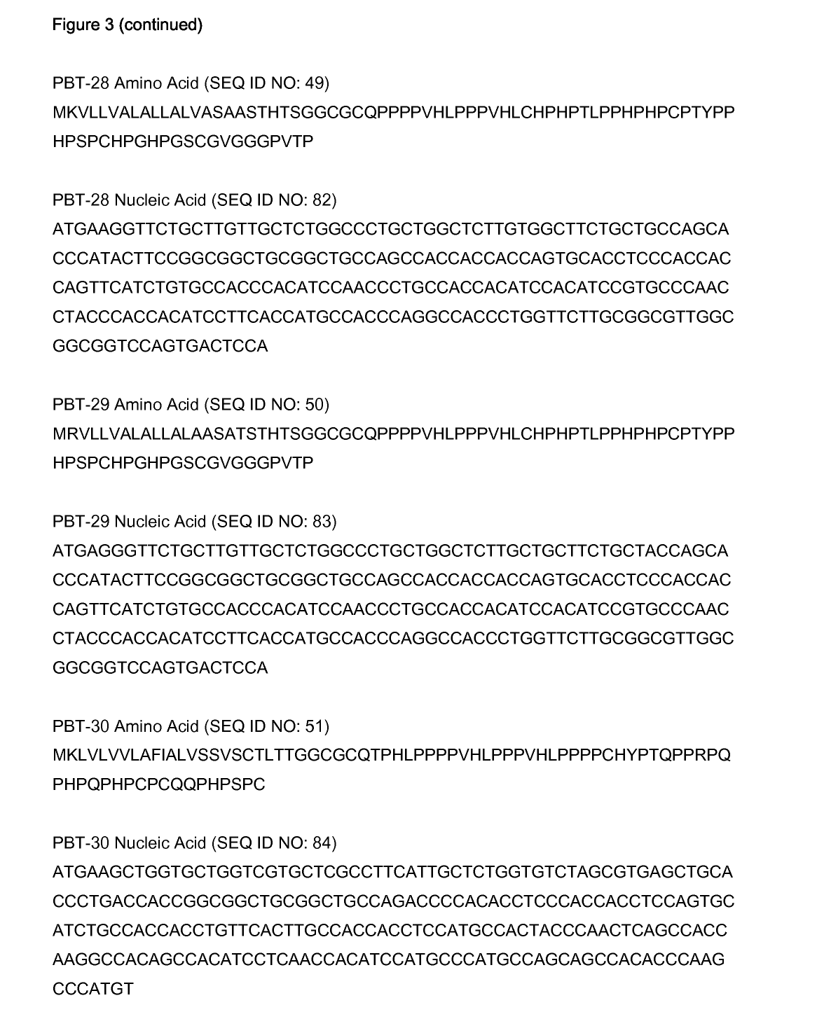

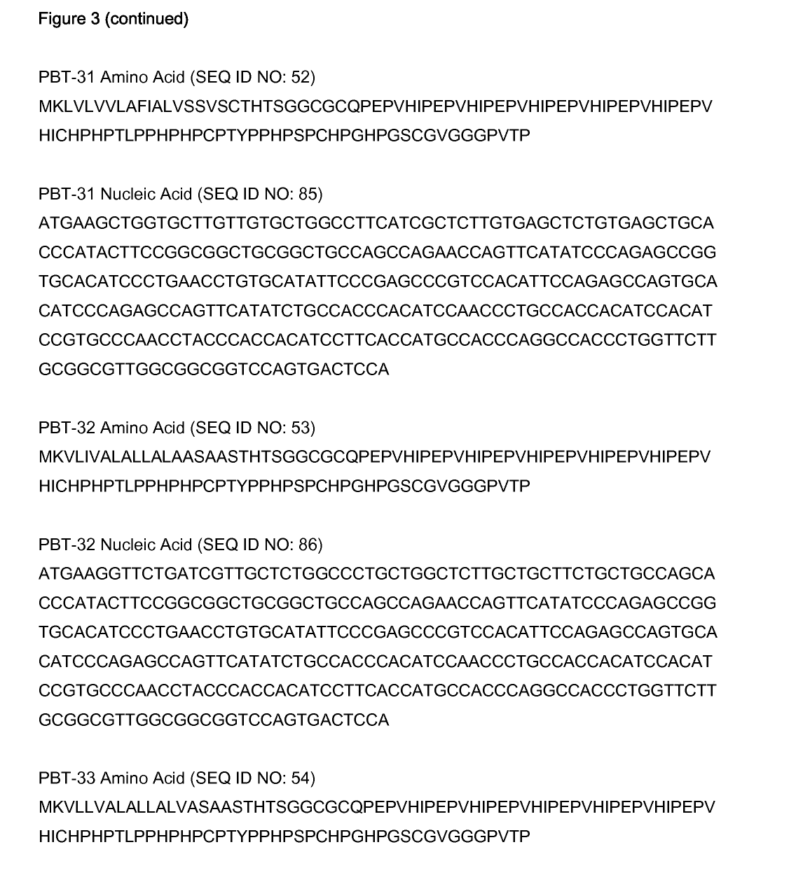

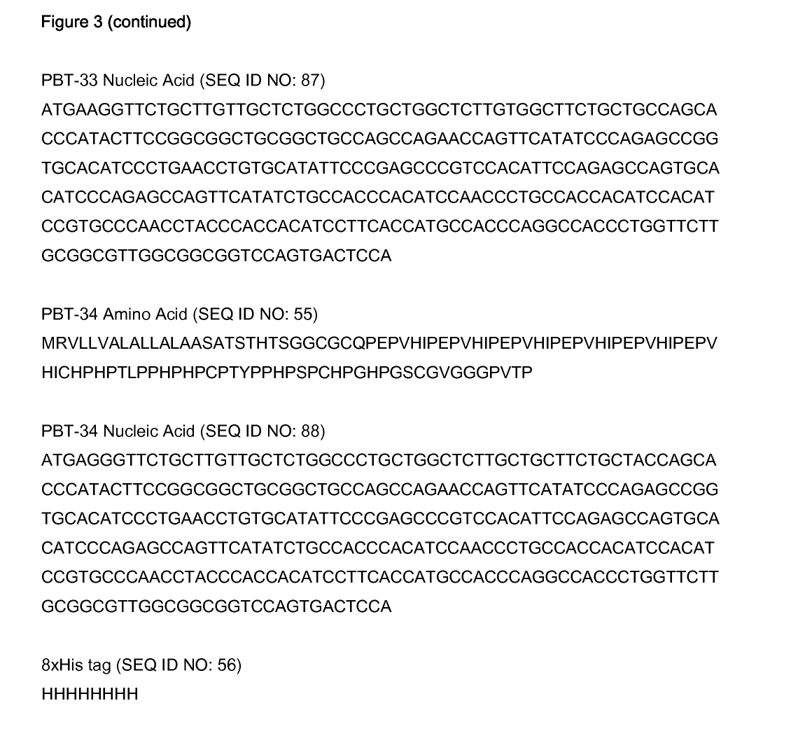

The wild-type 27 kDa γ-zein sequence region shown to drive protein body formation is comprised of the first 111 amino acids of the 27 kDa γ-zein protein. This region is called a protein body tag and includes four domains: the N-terminal signal peptide, a spacer region, a repeat domain comprising 7 repeats of the sequence PPPVHL (SEQ ID NO: 8), and a proline-rich domain referred to as the Pro-X domain. A depiction of these domains is shown in FIG. 1.

Prolamins are one of the four major classes of seed storage proteins which also include albumins, globulins, and glutelins. In certain cases, some storage proteins contain a repeat domain consisting of repeat units that are not conserved among different storage proteins. For example, cowpea glutelin-2 contains the repeat unit PEPVHI (SEQ ID NO: 11) while the 27 kDa γ-zein and γ-kafirin contain the repeat units of PPPVHL (SEQ ID NO: 8 or 10). This repeat domain is inserted within other regions that are rich in cysteine residues. These cysteine residues form disulfide bonds that likely contribute to protein body assembly. (Pompa et al., 2006, Plant Cell 18: 2608-2621). In this context, using site-directed mutagenesis of cysteine residues in the N-terminal proline-rich domain of γ-zein (Zera), Llop-Tous et al. have shown that the N-terminal cysteine residues Cys⁷ and Cys⁹ are essential for protein body oligomerization. (Llop-Tous et al., 2010, J Biol. Chem. 285 (46): 35633-44).

The term “protein bodies” refers to endoplasmic reticulum (ER)-derived or vacuole-derived protein aggregates surrounded by a membrane. Protein bodies are organelles that stably accumulate large amounts of storage proteins in seeds (Torrent et al., 2009, BMC Biology 7: 1-14). In cereals, protein bodies are formed in the ER lumen of endosperm cells and contain prolamin proteins. In maize, the 27 kDa γ-zein protein is located at the periphery of the protein body and surrounds aggregates of other proteins, including α-zein and 5-zein. (Torrent et al., 2009, Methods in Molecular Biology, Recombinant Proteins in Plants. Vol. 483, pp. 193-208). Protein bodies are normally formed in seed, but transgenic expression of the proline-rich N-terminal domain of γ-zein can induce the formation of protein body-like structures in non-seed tissues of Arabidopsis and tobacco. (Torrent et al., 2009, BMC Biology 7: 1-14). As used herein, the term “protein bodies” refers to protein bodies formed in seed as well as similar structures formed in other tissues. Protein bodies are described, for example, in Vitale et al. (2004, Plant Phys. 136: 3420-3426) and Loussert et al. (2008, J. Cereal Sci 47: 445-456).

A “protein body tag” is a polypeptide that induces the formation of protein bodies and/or targets a protein to a protein body in cells, tissues, or organisms. For example, a protein body tag may be fused to a protein of interest to target the protein of interest to the protein body. Protein body tags are comprised of a signal peptide, a spacer domain, a repeat domain, and a Pro-X domain. “Signal peptide” refers to the amino terminal extension of a polypeptide, which is translated in conjunction with the polypeptide forming a precursor peptide and which directs its entry into a secretory pathway. The “repeat domain” is a polypeptide domain comprising one or more amino acid repeat units derived from or homologous to the repeat regions of prolamin proteins. Examples of repeat units are shown in SEQ ID NO: 8, 10 and 11. The repeat domain of prolamin proteins occurs between the signal peptide and the Pro-X domain (Geli et al., 1994, Plant Cell 6: 1911-1922). The “Pro-X domain” is derived from the Pro-X region (also referred to as the P—X region), a proline-rich linker region found between the repeat region and the cysteine-rich C-terminal domain of prolamin proteins (Geli et al., 1994, Plant Cell 6: 1911-1922). A Pro-X domain may contain the entire Pro-X region or a fragment thereof. The spacer domain is located between the signal peptide and the repeat region. As an example, the domain structure of the 27 kDa γ-zein polypeptide is shown in FIG. 1.

As used herein, the term “wild-type variety” refers to a group of plants that are analyzed for comparative purposes as a control, wherein the wild-type variety plant is identical to the transgenic plant (plant transformed with an isolated polynucleotide in accordance with the invention) with the exception that the wild-type variety plant has not been transformed with a polynucleotide of the invention. The term “wild-type” as used herein refers to a plant cell, seed, plant component, plant part, plant tissue, plant organ, or whole plant that has not been genetically modified with a polynucleotide in accordance with the invention.

The term “modified” as applied to a nucleotide or amino acid molecule refers to a nucleotide or amino acid molecule having a sequence that has been changed to have a sequence different than the corresponding molecule as found in a wild-type plant, plant cell, seed, plant component, plant tissue, or plant organ.

The term “heterologous” refers to material (nucleic acid or protein) which is obtained from or derived from different source organisms, or, from different genes or proteins in the same source organism. Thus, a first domain that is “heterologous to” a second domain is obtained from or derived from a different nucleotide or polypeptide than the second domain. The heterologous domains may be derived from nucleotides or polypeptides from the same source species or from nucleotides or polypeptides from different species.

A modified protein body tag of the invention comprises four domains normally present in a wild-type protein body tag: a signal peptide; a spacer; a repeat domain comprising one or more repeat units; and a Pro-X domain, where, in one embodiment, at least one repeat unit of the repeat domain is heterologous to the Pro-X domain. In another embodiment, the signal peptide is from a different protein from the same species as the Pro-X domain. In yet another embodiment, at least one of the domains but not all of said domains is from a γ-kafirin protein. In another embodiment, the spacer is heterologous to the repeat domain or the Pro-X domain. In a further embodiment, at least one domain is from a γ-kafirin protein and the repeat domain has a different number of repeat units than a wild-type γ-kafirin repeat domain. Examples of repeat units are provided in SEQ ID NO: 8, 10 and 11. In one embodiment, the repeat domain comprises at least one but fewer than seven repeat units of the 27 kDa γ-zein protein (SEQ ID NO: 8). In another embodiment, the repeat domain may comprise one or more repeat units of SEQ ID NO: 10. In a further embodiment, the spacer may be heterologous to the repeat domain or the Pro-X domain.

In all instances, the modified protein body tag should retain the ability to direct a protein of interest to protein bodies in a cell.

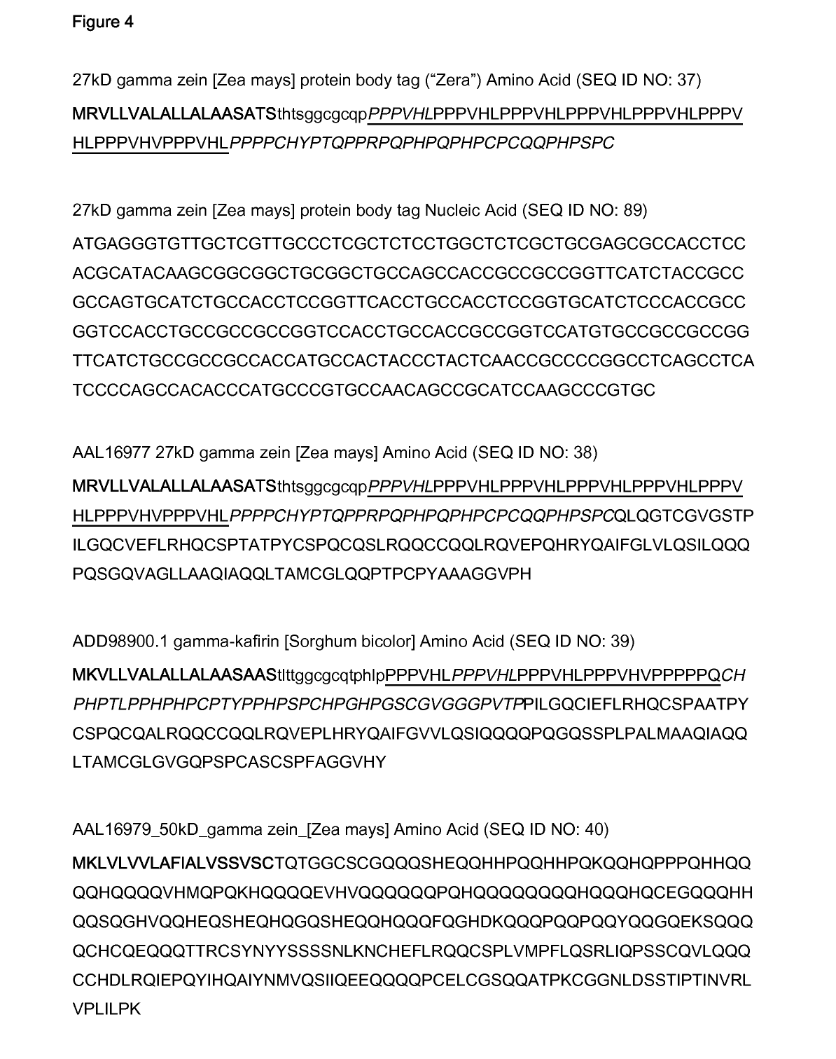

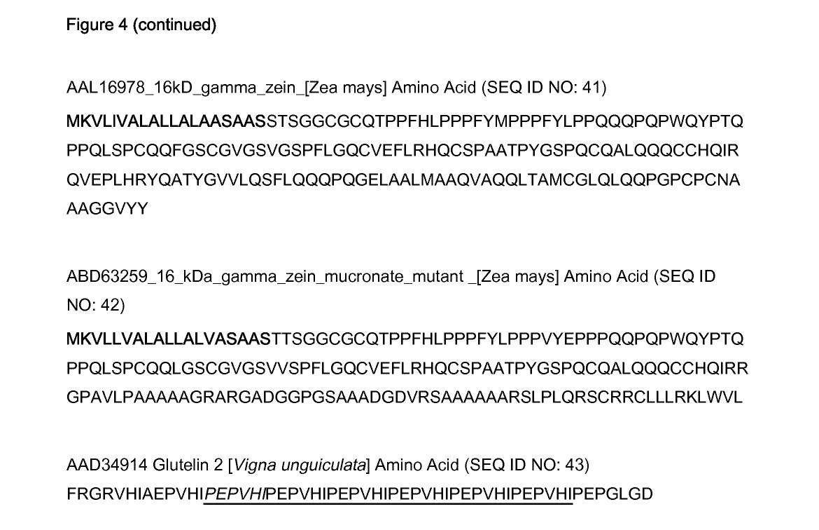

In some embodiments, the four domains are obtained from prolamins. In another embodiment, at least one of the domains or part thereof is obtained from a γ-zein protein, or homologs thereof. Prolamins suitable for the invention or from which one or more of the domains of a modified protein body tag may be derived include, but are not limited to: 16 kDa γ-zein (SEQ ID NO: 41 and 42), 27 kDa γ-zein (SEQ ID NO: 38), 50 kDa γ-zein (SEQ ID NO: 40), and γ-kafirin proteins (for example, SEQ ID NO: 39), for example, as shown in FIG. 4. The four domains may also be derived from other seed storage proteins, such as cowpea glutelin-2 (SEQ ID NO: 43). The 27 kDa γ-zein protein, 50 kDa γ-zein protein, 16 kDa γ-zein proteins, γ-kafirin, and cowpea γ-zein ortholog (cowpea glutelin-2) are considered γ-zein protein homologs.

Examples of domains from which modified protein body tags may be derived are presented in Table 1.

TABLE 1

| | | SEQ |
|---|---|---|
| | | ID |
| Signal peptide | Source | NO: |
| MKLVLVVLAFIALVSSVSC | 50 kDa γ-Zein | 1 |
| | (AAL16979) | |
| MKVLIVALALLALAASAAS | 16 kDa γ-Zein | 2 |
| | (AAL16978) | |
| MKVLLVALALLALVASAAS | 16 kDa γ-Zein | 3 |
| | (ABD63259) | |
| MRVLLVALALLALAASATS | 27 kDa γ-Zein | 4 |
| | (AAL16977) | |
| MKVLLVALALLALAASAAS | γ-Kafirin | 5 |
| | (ADD98900) | |
| MKTNLFLFLIFSLLLSLSSA | Basic | 9 |
| | Endochitinase b | |
| | (Haseloff et al., | |
| | PNAS (1997) | |
| | 94: 2122-2127) | |
| Spacer | | |
| THTSGGCGCQP | 27 kDa γ-Zein | 6 |
| | (AAL16977) | |
| TLTTGGCGCQTPHLP | γ-Kafirin | 7 |
| | (ADD98900) | |
| Repeats | | |
| PPPVHL | 27 kDa γ-Zein | 8 |
| | (AAL16977) | |
| PPPVHL | γ-Kafirin | 10 |
| | (ADD98900) | |
| PEPVHI | Cowpea Glutelin | 11 |
| | 2 (AAD34914) | |
| ProX Domain | | |
| PPPPCHYPTQPPRPQPHPQPHPCPCQQPHPSPC | 27 kDa γ-zein | 12 |
| | (AAL16977) | |
| CHPHPTLPPHPHPCPTYPPHPSPCHPGHPGSCGVGGG | γ-Kafirin | 13 |
| PVTP | (ADD98900) | |

In one embodiment, the modified protein body tag comprises a signal peptide, a spacer domain, a repeat domain comprising one or more repeat units, and a Pro-X domain, wherein the signal peptide comprises the sequence of SEQ ID NO: 1, SEQ ID NO: 2, SEQ ID NO: 3, SEQ ID NO: 4, or SEQ ID NO: 5; wherein the repeat domain comprises one or more repeat units of the sequence SEQ ID NO: 8, SEQ ID NO: 10, or SEQ ID NO: 11; wherein the Pro-X domain comprises the sequence of SEQ ID NO: 12 or SEQ ID NO: 13; and wherein the spacer domain comprises the sequence of SEQ ID NO: 6 or SEQ ID NO: 7.

In yet a further embodiment, at least one domain of the modified protein body tag but not all of the domains is from a γ-kafirin protein. For example, at least one of the domains of the modified protein body tag is substituted with the corresponding domain from a different species or from a different gene or protein of the same or different species such that one or more γ-kafirin domains are associated with one or more non-γ-kafirin domains. In another embodiment, at least one domain of the modified protein body tag is from a γ-kafirin protein and the repeat domain has a different number of repeat units than a wild-type γ-kafirin repeat domain, for example, the repeat domain comprises one or more repeat units of SEQ ID NO: 10.

As defined herein, the term “nucleic acid” and “polynucleotide” are interchangeable and refer to RNA or DNA that is linear or branched, single or double stranded, or a hybrid thereof. The term also encompasses RNA/DNA hybrids. An isolated nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequences located at both the 3′ and 5′ ends of the coding region of a gene, it may be preferable to remove the sequences which naturally flank the coding region in its naturally occurring replicon. Unless otherwise indicated, a particular nucleic acid sequence also implicitly encompasses conservatively modified variants thereof such as degenerate codon substitutions and complementary sequences as well as the sequence explicitly indicated. In one embodiment, the invention relates to nucleic acids which encode the modified protein body tags, the complement of these nucleic acids, and nucleic acids which hybridize to these nucleic acids. In certain embodiments, nucleic acids and proteins can be isolated.

The terms “protein,” “peptide” and “polypeptide” are used interchangeably herein.

“Expression cassette” as used herein means a DNA molecule which includes sequences capable of directing expression of a particular nucleotide sequence (e.g., which codes for a protein of interest) in an appropriate host cell, including regulatory sequences such as a promoter operably linked to a nucleotide sequence of interest, optionally associated with termination signals and/or other regulatory elements. An expression cassette may also comprise sequences required for proper translation of the nucleotide sequence. The coding region of the expression cassette usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. An expression cassette may be assembled entirely extracellularly (e.g., by recombinant cloning techniques). The expression of the nucleotide sequence in the expression cassette may be under the control of a promoter.

Selection of promoters will depend on several factors, such as the trait of interest and/or on the type of host cell. For example, for increased biomass or silage quality, constitutive promoters may be used. For seed traits such as increased seed yield or increased seed protein content, seed-specific promoters may be used.

The terms “regulatory sequence”, “regulatory element”, “control sequence” are all used interchangeably herein and are to be taken in a broad context to refer to any sequence that controls or is capable of effecting expression of the sequences to which they are ligated in a cell. Regulatory sequences may include promoter, terminators, enhancers, and the like. An example of a regulatory sequence is a promoter, which typically refers to a nucleic acid control sequence located upstream from the transcriptional start of a gene and which is involved in recognizing and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers), which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” is a type of regulatory element, which mediates the expression of a coding sequence in plant cells. Accordingly, a plant promoter need not be of plant origin, but may originate from viruses or micro-organisms, for example from viruses which attack plant cells, or, it might be a synthetic promoter designed by man. The “plant promoter” can also originate from a plant cell, e.g. from the plant which is transformed with the nucleic acid sequence to be expressed. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) so long as it does not interfere with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, including promoters from heterologous organisms. For expression in plants, the nucleic acid molecule, as described above, can be linked operably to or comprise a suitable promoter which expresses the gene at a desired point in time and/or with a selected spatial expression pattern.

The term “operably linked” as used herein refers to a functional linkage between two sequences, for example, between a promoter sequence and a gene of interest such that the promoter sequence is able to initiate transcription of the gene of interest. The term “operably linked” may also refer, for example, to a functional linkage between a protein body tag and a protein of interest for targeting and/or accumulation of the protein of interest to a protein body.

As known in the art, promoters may be constitutive, inducible, developmental stage-preferred, developmentally-regulated, cell type-specific or preferred, tissue-specific or preferred, or organ-specific or preferred. Non-limiting examples of constitutive promoters include the Actin (McElroy et al, Plant Cell, 2: 163-171 1990), HMGP (WO 2004/070039), CAMV 35S (Odell et al, Nature, 313: 810-812, 1985), CaMV 19S (Nilsson et al., Physiol. Plant. 100:456-462, 1997), GOS2 (de Pater et al, Plant J November; 2(6):837-44, 1992, WO 2004/065596), Ubiquitin (Christensen et al, Plant Mol. Biol. 18: 675-689, 1992), Rice cyclophilin (Buchholz et al, Plant Mol. Biol. 25(5): 837-43, 1994), Maize H3 histone (Lepetit et al, Mol. Gen. Genet. 231:276-285, 1992), Alfalfa H3 histone (Wu et al. Plant Mol. Biol. 11:641-649, 1988), Actin 2 (An et al, Plant J. 10(1); 107-121, 1996), 34S FMV (Sanger et al., Plant. Mol. Biol., 14, 1990: 433-443), Rubisco small subunit (U.S. Pat. No. 4,962,028), OCS (Leisner (1988) Proc Natl Acad Sci USA 85(5): 2553), SAD1 (Jain et al., Crop Science, 39 (6), 1999: 1696), SAD2 (Jain et al., Crop Science, 39 (6), 1999: 1696), nos (Shaw et al. (1984) Nucleic Acids Res. 12(20):7831-7846), V-ATPase (WO 01/14572), Super promoter (WO 95/14098), and G-box protein (WO 94/120150) promoters, and the like. In one embodiment, the promoter is from the Oryza sativa (rice) caffeoyl CoA-O-methyltransferase (OsCCoAMT) gene (WO 06/084868, which is hereby incorporated by reference in its entirety). Choice of promoter will depend on several factors, such as the type of host cell. An organ-specific or tissue-specific promoter is one that is capable of preferentially initiating transcription in certain organs or tissues, such as the leaves, roots, seed tissue, green tissue, meristem, etc. For example, a “seed-specific promoter” is a promoter that is transcriptionally active predominantly in plant seeds, substantially to the exclusion of any other parts of a plant, while still allowing for any leaky expression in other plant parts. Examples of seed-specific promoters are provided in Qing Qu and Takaiwa (Plant Biotechnol. J. 2, 113-125, 2004), which disclosure is incorporated by reference herein as if fully set forth. Further non-limiting examples of seed-specific promoters include the seed-specific gene (Simon et al., Plant Mol. Biol. 5: 191, 1985; Scofield et al., J. Biol. Chem. 262: 12202, 1987; Baszczynski et al., Plant Mol. Biol. 14: 633, 1990), Brazil Nut albumin (Pearson et al., Plant Mol. Biol. 18: 235-245, 1992), legumin (Ellis et al., Plant Mol. Biol. 10: 203-214, 1988), glutelin (rice) (Takaiwa et al., Mol. Gen. Genet. 208: 15-22, 1986; Takaiwa et al., FEBS Letts. 221: 43-47, 1987), zein (Matzke et al Plant Mol Biol, 14(3):323-32 1990), napA (Stalberg et al, Planta 199: 515-519, 1996), wheat LMW and HMW glutenin-1 (Mol Gen Genet. 216:81-90, 1989; NAR 17:461-2, 1989), wheat SPA (Albani et al, Plant Cell, 9: 171-184, 1997), wheat a, 13, γ-gliadins (EMBO J. 3:1409-15, 1984), barley 1 tr 1 promoter (Diaz et al. (1995) Mol Gen Genet. 248(5):592-8), barley B1, C, D, hordein (Theor Appl Gen 98:1253-62, 1999; Plant J 4:343-55, 1993; Mol Gen Genet. 250:750-60, 1996), barley DOF (Mena et al, The Plant Journal, 116(1): 53-62, 1998), blz2 (EP99106056.7), synthetic promoter (Vicente-Carbajosa et al., Plant J. 13: 629-640, 1998), rice prolamin NRP33 (Wu et al, Plant Cell Physiology 39(8) 885-889, 1998), rice α-globulin Glb-1 (Wu et al, Plant Cell Physiology 39(8) 885-889, 1998), rice OSH1 (Sato et al, Proc. Natl. Acad. Sci. USA, 93: 8117-8122, 1996), rice α-globulin REB/OHP-1 (Nakase et al. Plant Mol. Biol. 33: 513-522, 1997), rice ADP-glucose pyrophosphorylase (Trans Res 6:157-68, 1997), maize ESR gene family (Plant J 12:235-46, 1997), sorghum α-kafirin (DeRose et al., Plant Mol. Biol. 32:1029-35, 1996), KNOX (Postma-Haarsma et al, Plant Mol. Biol. 39:257-71, 1999), rice oleosin (Wu et al, J. Biochem. 123:386, 1998), sunflower oleosin (Cummins et al., Plant Mol. Biol. 19: 873-876, 1992), PRO0117, putative rice 40S ribosomal protein (WO 2004/070039), PRO0136, rice alanine aminotransferase (unpublished), PRO0147, trypsin inhibitor ITR1 (barley) (unpublished), PRO0151, rice WSI18 (WO 2004/070039), PRO0175, rice RAB21 (WO 2004/070039), PRO005 (WO 2004/070039), PRO0095 (WO 2004/070039), α-amylase (Amy32b) (Lanahan et al, Plant Cell 4:203-211, 1992; Skriver et al, Proc Natl Acad Sci USA 88:7266-7270, 1991), cathepsin β-like gene (Cejudo et al, Plant Mol Biol 20:849-856, 1992), Barley Ltp2 (Kalla et al., Plant J. 6:849-60, 1994), Chi26 (Leah et al., Plant J. 4:579-89, 1994), and Maize B-Peru (Selinger et al., Genetics 149; 1125-38, 1998) promoters, and the like.

Plant gene expression can also be facilitated via an inducible promoter. An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or “pathogen-inducible” i.e. activated when a plant is exposed to various pathogens. Chemically inducible promoters are especially suitable if gene expression is desired in a time specific manner. Examples for such promoters are a salicylic acid inducible promoter (WO 95/19443), a tetracycline inducible promoter (Gatz et al. 1992, Plant J. 2:397-404) and an ethanol inducible promoter (WO 93/21334). Promoters responding to biotic or abiotic stress conditions are also suitable promoters such as the pathogen inducible PRP1-gene promoter (Ward et al., 1993, Plant Mol. Biol. 22:361-366), the heat inducible hsp80-promoter from tomato (U.S. Pat. No. 5,187,267), cold inducible alpha-amylase promoter from potato (WO 96/12814) or the wound-inducible pinll-promoter (EP 375091).

The term “terminator” encompasses regulatory elements which signal 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

“Vector” is defined to include, inter alia, any plasmid, cosmid, phage or Agrobacterium vector or binary vector in double or single stranded linear or circular form which may or may not be self transmissible or mobilizable, and which can transform prokaryotic or eukaryotic host cells either by integration into the cellular genome or exist extrachromosomally (e.g. an autonomous replicating plasmid with an origin of replication). Specifically included are shuttle vectors by which is meant a DNA vehicle capable, naturally or by design, of replication in two different host organisms, which may be selected from Actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

Cloning vectors can contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Cloning vectors also include vectors in which DNA can be introduced through homologous recombination, such as the GATEWAY® vectors (Invitrogen, see webpage at invitrogen.com).

Proteins of interest can be any protein which provides a trait of interest. Proteins of interest may include proteins involved in seed quality, seed yield, total yield, total biomass, nutritional value, protein and/or amino acid content, oil content, silage quality, feed quality, digestibility, early vigor, disease and insect resistance, and cold, heat and drought tolerance. Proteins of interest also include seed storage proteins including, but not limited to, albumins, prolamins, globulins, prolamins and glutelins. Proteins of interest may also include green fluorescent protein (GFP), DsRED, GUS, epidermal growth factor (EGF), a quality plant protein, or a protein that confers a desirable agronomic trait or of biopharmaceutical interest. Proteins of interest may also include markers, for example, that confer antibiotic or herbicide resistance, that introduce a new metabolic trait or that allow visual selection of a transgenic cell or organism.

In one embodiment, the invention encompasses nucleic acid molecules comprising a nucleic acid sequence encoding a modified protein body tag. In specific embodiments, the invention relates to a nucleic acid molecule comprising a nucleic acid sequence encoding a protein body tag comprising the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55, or functional variants thereof.

In a further embodiment, the invention also relates to a nucleic acid which comprises the sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88, or a functional variant thereof and which encodes a modified protein body tag.

In another embodiment, the invention also relates to expression cassettes comprising a nucleic acid molecule comprising a nucleic acid sequence encoding a modified protein body tag, at least one nucleic acid molecule encoding a protein of interest, and a regulatory sequence that drives expression in a host cell. In another embodiment, at least one nucleic acid molecule encoding a protein of interest may be operably linked to a regulatory sequence that drives expression in a plant cell. The regulatory sequence may comprise a promoter such as a seed-specific, constitutive, tissue-specific, ubiquitous, or developmentally regulated promoter.

In a further embodiment, the invention also encompasses polypeptides comprising the modified protein body tags. In certain embodiments, the polypeptide comprises the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55, or functional variants thereof.

Nucleotide sequences may be codon optimized to improve expression in heterologous host cells. Nucleotide sequences from a heterologous source are codon optimized to match the codon bias of the host. A codon consists of a set of three nucleotides, referred to as a triplet, which encodes a specific amino acid in a polypeptide chain or for the termination of translation (stop codons). The genetic code is redundant in that multiple codons specify the same amino acid, i.e., 61 codons encoding for 20 amino acids. Organisms exhibit preference for one of the several codons encoding the same amino acid, which is known as codon usage bias. The frequency of codon usage for different species has been determined and recorded in codon usage tables. Codon optimization replaces infrequently used codons present in a DNA sequence of a heterologous gene with preferred codons of the host, based on a codon usage tables. The amino acid sequence is not altered during the process. Codon optimization can be performed using gene optimization software, such as Leto 1.0 from Entelechon. Protein sequences for the genes to be codon optimized are back-translated in the program and the codon usage is selected from a list of organisms. Leto 1.0 replaces codons from the original sequence with codons that are preferred by the organism into which the sequence will be transformed. The DNA sequence output is translated and aligned to the original protein sequence to ensure that no unwanted amino acid changes were introduced.

In addition to codon optimization of a sequence from a heterologous source, gene optimization entails further modifications to the DNA sequence to optimize the gene sequence for expression without altering the protein sequence. The Leto 1.0 program can also be used to remove sequences that might negatively impact gene expression, transcript stability, protein expression or protein stability, including but not limited to, transcription splice sites, DNA instability motifs, plant polyadenylation sites, secondary structure, AU-rich RNA elements, secondary ORFs, codon tandem repeats, long range repeats. This can also be done to optimize gene sequences originating from the host organism. Another component of gene optimization is to adjust the G/C content of a heterologous sequence to match the average G/C content of endogenous genes of the host.

For example, to provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) eliminate sequences that cause destabilization, inappropriate polyadenylation, degradation and termination of RNA, or that form secondary structure hairpins or RNA splice sites; or 5) eliminate antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

In some embodiments of the invention, the nucleic acid molecule encoding the modified protein body tag is codon optimized. The nucleic acid sequence may be codon optimized for any host cell in which it is expressed. In one embodiment, the nucleic acid sequence is codon optimized for maize. In further embodiments, the nucleic acid sequence may also be codon optimized for other plant species including, but not limited to, tobacco, Arabidopsis, rice, wheat, barley, soybean, canola, rapeseed, cotton, sugarcane, or alfalfa.

The nucleotide and amino acid sequences of the invention include both the naturally occurring sequences as well as mutant (variant) forms. Modification of a nucleotide or amino acid sequence includes the production of variants of that sequence. Such variants will continue to possess the desired activity of the non-variant sequences, i.e. functional variants, for example, with protein body tags, induction of protein body-like structures. The term “variant” with respect to a molecule (e.g., a polypeptide or nucleic acid sequence such as, for example, a protein body tag of the invention and/or a protein of interest) is intended to mean substantially similar sequences in which the activity is retained in whole or in part. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide and amino acid sequence variants of the invention will have at least 40, 50, 60, to 70%, e.g., preferably 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the native (wild type or endogenous) nucleotide or amino acid sequence. The protein body tags of the invention, or one or more of the domains thereof, may be variants of the wild-type sequence, provided they retain the ability of directing and/or accumulation of a protein of interest to protein bodies in cells. A modified protein body tag may also contain domains from the same species but have one or more insertions, deletions, or substitutions in one or more of these domains.

Modification of a nucleotide or amino acid sequence also includes substitution of a fragment of that sequence with a corresponding sequence from a related gene or protein. For example, in one embodiment, modification of a protein body tag may be achieved by substituting one of the domains with the corresponding domain from another protein from the same or from a different species. A modified gene or protein may comprise regulatory sequences and coding sequences that are derived from different sources, or comprise regulatory sequences and coding sequences derived from the same source, but arranged in a manner different from that found in nature. The term also includes non-naturally occurring multiple copies of a naturally occurring DNA or protein sequences. A modified gene may also contain insertions, deletions, or substitutions of one or more nucleotides relative to the nucleotide sequence found in nature. A modified protein may contain insertions, deletions, or substitutions of one or more amino acid residues relative the amino acid sequence found in nature.

As used herein, “sequence identity” or “identity” in the context of two nucleic acid or polypeptide sequences refers to the residues in the two sequences that are the same when aligned for maximum correspondence over a specified comparison window. As used herein, “percentage of sequence identity” means the value determined by comparing two optimally aligned sequences over a specified comparison window.

Methods of alignment of sequences for comparison and calculation of percent sequence identity are well known in the art. For example, the percent sequence identity may be determined with the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide. Sequence alignments and calculation of percent sequence identity may also be performed with CLUSTAL (see website at ebi.ac.uk/Tools/clustalw2/index.html) the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))), which are part of the GCG software packet [Genetics Computer Group, 575 Science Drive, Madison, Wis., USA 53711 (1991)].

Methods of identifying homologous sequences with sequence similarity to a reference sequence are known in the art. For example, software for performing BLAST analyses for identification of homologous sequences is publicly available through the National Center for Biotechnology Information (see website at ncbi.nlm.nih.gov). PSI-BLAST (in BLAST 2.0) can also be used to perform an iterated search that detects distant relationships between molecules. When utilizing BLAST or PSI-BLAST, the default parameters of the respective programs (e.g. BLASTN for nucleotide sequences, BLASTX for proteins) can be used. See ncbi.nlm.nih.gov website. Alignment may also be performed manually by inspection. These methods may be used, for example, to identify prolamin sequence homologs for the assembly of protein body tags (see Example 1).

Nucleic acid molecules corresponding to functional variants, homologs, analogs, and orthologs of polypeptides can be isolated based on their identity to said polypeptides. The polynucleotides encoding the respective polypeptides or primers based thereon can be used as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

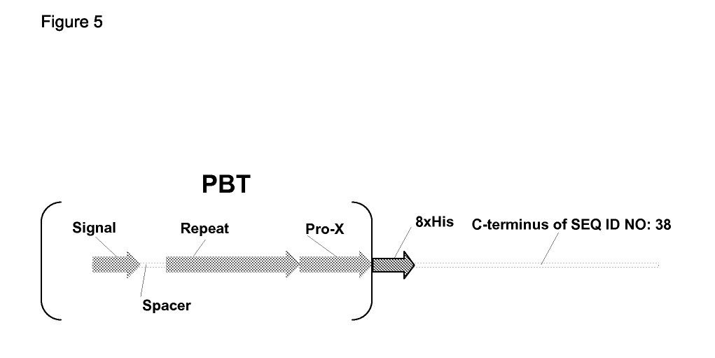

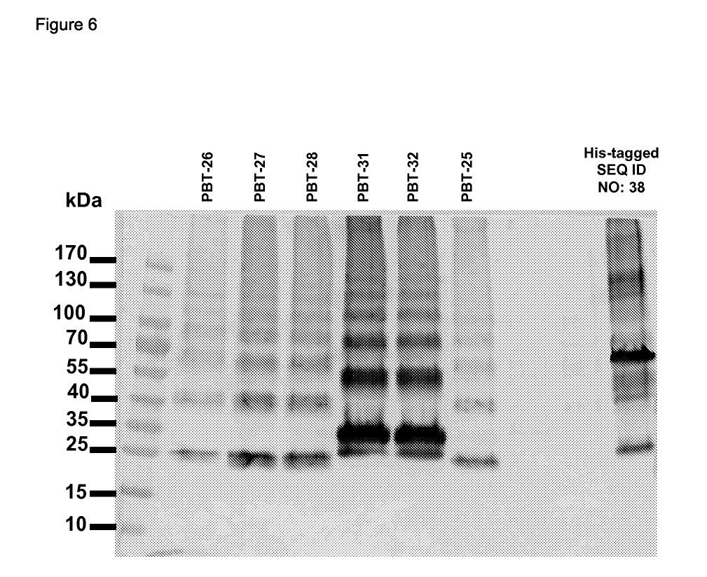

The invention also relates to fusion proteins comprising a first polypeptide comprising a modified protein body tag and a second polypeptide comprising at least one protein of interest. An example of such a fusion protein has the amino acid sequence depicted in FIG. 5.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” may also include parts of plants, such as pollen, flowers, kernels, ears, cobs, leaves, husks, stalks, and the like. The term “plant” also encompasses plant cells, plant protoplasts, plant cell tissue cultures, callus tissue, embryos, meristematic regions, gametophytes, sporophytes, pollen and microspores, gamete producing cells, and a cell that regenerates into a whole plant, again wherein each of the aforementioned comprises the gene/nucleic acid of interest.

Plants that are particularly useful in the methods of the invention include microalgae and all plants which belong to the superfamily Viridiplantae. Examples of microalgae include Cyclotella cryptica, Navicula saprophila, Synechococcus 7002 and Anabaena 7120, Chlorella protothecoides, Dunaliella salina, Chlorella spp, Dunaliella tertiolecta, Gracilaria, Sargassum, Pleurochrisis carterae, Laminaria 3840 hyperbore, Laminaria saccharina, Gracialliaria, Sargassum, Botryccoccus braunii, and Arthospira platensis. Plants which belong to the superfamily Viridiplantae include monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others. Especially preferred are A. thaliana, Nicotiana tabacum, rice, oilseed rape, canola, soybean, corn (maize), cotton, sugarcane, alfalfa, sorghum, and wheat.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The invention also relates to a vector, plant cell, plant tissue, plant or parts thereof, progeny or seed thereof comprising a nucleic acid encoding a modified protein body tag. In some embodiments, the vectors, plant cells, plant tissue, plants or parts thereof, progeny or seed thereof comprise expression cassettes comprising the nucleic acids encoding modified protein body tags and at least one nucleic acid molecule encoding a protein of interest operably linked to a regulatory sequence that permits expression in a host cell. The regulatory sequence may comprise a promoter such as a seed-specific, constitutive, tissue-specific, ubiquitous, or developmentally regulated promoter. The invention further relates to a transgenic plant cell, plant, or part thereof comprising in its genome at least one stably incorporated expression cassette as described above and the transgenic seed or transgenic progeny of these plants. The plant cell, plant tissue, plant or part thereof, progeny or seed thereof may be obtained from any plant, including but not limited to, tobacco, Arabidopsis, maize, rice, wheat, barley, soybean, canola, rapeseed, cotton, sugarcane, or alfalfa.

In one embodiment, the plant cell, plant tissue, plant or part thereof comprises a nucleic acid molecule encoding the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55, or variants thereof. In yet a further embodiment, the plant cell, plant tissue, plant or part thereof comprises one or more nucleic acid molecule encoding the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and/or SEQ ID NO: 13, or functional variants thereof.

A transgene refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. Endogenous gene refers to a native gene in its natural location in the genome of an organism. A foreign gene refers to a gene not normally found in the host organism but that is introduced by gene transfer.

As used herein, the term “transgenic” refers to any cell, organism, plant, plant cell, callus, plant tissue, or plant part, that contains the expression cassette described above. In one embodiment, the expression casette is stably integrated into a chromosome or stable extra-chromosomal element, so that it is passed on to successive generations.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Advantageously, any of several known transformation methods may be used to introduce the gene of interest into a suitable host cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. A polynucleotide may be introduced into a plant cell by any means, including transfection, transformation or transduction, electroporation, particle bombardment, agroinfection, and the like.

Transgenic plants, including transgenic crop plants, may be produced via Agrobacterium-mediated transformation. In the case of corn transformation, methods are described in WO2006/136596, U.S. Pat. No. 5,591,616, Ishida et al. (Nat. Biotechnol 14(6): 745-50, 1996) and Frame et al. (Plant Physiol 129(1): 13-22, 2002), which disclosures are incorporated by reference herein as if fully set forth. Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. These methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38. Methods for Agrobacterium-mediated transformation of Arabidopsis are provided, for example, in Clough, S J and Bent A F (1998) The Plant J. 16, 735-743. Additionally, methods of transformation are provided in Peng et al., 2006 (WO2006/136596) which is incorporated herein by reference in its entirety.

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

The term “increased expression” or “overexpression” as used herein means any form of expression that is additional to the wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. For example, for maize, introns Adh1-S intron 1, 2, and 6, the Bronze-1 intron are known in the art. For general information see: The Maize Handbook, Chapter 116, Freeling and Walbot, Eds., Springer, N.Y. (1994).

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Non-limiting examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to BASTA®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of color (for example β-glucuronidase, GUS or 3-galactosidase with its colored substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids may employ techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

The invention also relates to a transformed plant cell, plant or part thereof comprising in its genome at least one stably incorporated expression cassette comprising a first nucleotide sequence encoding a protein body tag and at least one second nucleotide sequence encoding a protein of interest operably linked to a regulatory sequence that drives expression in a plant cell.

Host cell systems may be used for evaluating protein body targeting and/or formation. In one embodiment, the host cell system may comprise one or more host cells which comprise a) a nucleic acid molecule comprising a nucleic acid sequence encoding a protein body tag and b) at least one nucleic acid molecule encoding a protein of interest operably linked to a regulatory sequence that drives expression in the host cell. The regulatory sequence may comprise a promoter, for example, a constitutive promoter. The nucleic acid molecule encoding the protein of interest may comprise one or more reporter genes. The host cells may be any type of cell including plant, animal, or insect cells. In one embodiment, the host cell is a plant cell. In a further embodiment, the plant cells are obtained from tobacco, Arabidopsis, or maize. In another embodiment, the maize cell is a Black Mexican Sweetcorn (BMS) maize cell. Transgenic host cell culture methods are known in the art and are provided in the Examples and in Torrent et al. 2009, BMC Biology 7: 1-14. Methods for evaluating the host cell for protein body formation and/or expression of proteins of interest are provided for example in Geli et al., 1994, Plant Cell 6: 1911-1922; Torrent et al., 2009, BMC Biology 7: 1-14; and Torrent et al., 2009, Methods in Molecular Biology 483: 193-208.

Methods of the invention also include a method for designing a protein body tag of reduced allergenicity, which comprises
a) providing amino acid sequences which encode the signal peptide domain, the spacer domain, repeat domain, and Pro-X domain of a protein body tag, which sequences together define the amino acid sequence of a designed protein body tag;
b) comparing the sequence of said designed protein body tag to a database of allergenic proteins to identify areas of homology, if any, between the designed protein body tag and the proteins contained in the database, which areas of homology signify potential allergenicity; and
c) identifying designed protein body tags having no or few areas of homology which signify potential allergenicity as indicated by said comparison.

Allergen sequence databases for screening protein body tags include the Allergen Nomenclature database of the International Union of Immunological Societies (IUIS)

Allergen Nomenclature Sub-Committee (website at allergen.org) (Hoffman et al., 1994, Bull. of the World Health Organization 72: 796-806); the Allergen Online database maintained by the Food Allergy Research and Resource Program of the University of Nebraska (website at allergenonline.org); and the Structural Database of Allergen Proteins (SDAP) (fermi.utmb.edu/SDAP/sdap_ver.html) (Ivanciuc et al., 2003, Nucl. Acids Res. 31: 359-362). Once the screening is completed, a designed protein body tag having no or few areas of homology between the designed protein body tag and the proteins contained in the database (as the databases exist currently or as altered or expanded in the future), which areas of homology signify potential allergenicity is selected and used in further constructs and experimentation. For example, the areas of potential allergenicity may include areas defined by 8 contiguous amino acids or defined by 80 contiguous amino acids.

The invention also encompasses the modified protein body tags obtained by the screening methods described above and nucleic acid molecules encoding them. In one embodiment, the domains of said protein body tag are selected such that at least one domain is heterologous to at least one other domain. In another embodiment, the protein body tag identified as a designed protein body tag has a non-wild-type sequence. In a further embodiment, the protein body tag identified as a designed protein body tag comprises the modified protein body tag as described herein, for example, which comprise a signal peptide domain, a spacer domain, a repeat domain comprising one or more repeat units, and a Pro-X domain, wherein
(i) at least one repeat unit of the repeat domain is heterologous to the Pro-X domain,
(ii) the signal peptide is from a different protein from the same species as the Pro-X domain,
(iii) at least one of the domains but not all of said domains is from a γ-kafirin protein, and/or,
(iv) the spacer is heterologous to the repeat domain or the Pro-X domain.

Methods of the invention include a method for evaluating protein body targeting and/or formation and/or accumulation of a protein of interest, comprising a) culturing a transgenic host cell comprising an expression cassette which comprises a nucleotide sequence encoding a designed or modified protein body tag and at least one nucleic acid molecule encoding a protein of interest operably linked to a regulatory sequence that drives expression in the host cell; and b) evaluating the transgenic host cell for protein body formation and/or for expression of the protein of interest. Expression cassettes for the evaluation of protein body formation may include for example, a Histidine tag and/or other epitope tag in order to aid the evaluation process in the transgenic system. Methods for evaluating transgenic plants or plant cells for protein body formation and/or expression of proteins of interest are provided, for example, in Geli et al., 1994, Plant Cell 6: 1911-1922; Torrent et al., 2009, BMC Biology 7: 1-14; and Torrent et al., 2009, Methods in Molecular Biology 483: 193-208.

A protein body tag evaluated in a host cell can be used to generate a transgenic plant comprising the protein body tag. In one embodiment, the transgenic plant is generated directly from the cell culture used for evaluation. In another embodiment, the protein body tag may be excised from the expression cassette used for evaluation in the host cell and cloned into a new expression cassette comprising a different protein of interest. In another embodiment, the protein body tag is provided in an expression cassette with one or more proteins of interest and appropriate regulatory elements for expression. Transgenic plants comprising these expression cassettes may be generated.

In a further embodiment, the invention provides a method of producing a transgenic plant which targets a protein of interest to a protein body, the method comprising:
a) transforming a plant cell with an expression cassette comprising
   i) a first nucleotide sequence comprising a nucleotide sequence encoding a modified protein body tag; and
   ii) a second nucleotide sequence encoding a protein of interest; and
b) regenerating a transgenic plant from the plant cell.

The plant or plant cell optionally further comprises at least one other nucleotide sequence encoding another protein of interest. This further protein of interest may be overexpressed or downregulated by methods known in the art and may comprise another storage or seed protein such as the prolamin α-zein.

The modified protein body tags may improve protein body formation and/or improve targeting and/or accumulation of proteins to protein bodies relative to wild-type protein body tags. In a further embodiment, the invention relates to a method for improving protein body formation and/or improving targeting and/or accumulation of proteins to protein bodies in a transgenic plant or plant cell relative to a corresponding wild-type plant or plant cell comprising growing or culturing a transgenic plant cell, plant or part thereof which comprises a modified protein body tag of the invention.

The nucleic acid sequences can be used to alter or increase the levels of one or more protein of interest in the protein bodies of a transgenic plant, such as A. thaliana, Nicotiana tabacum, rice, oilseed rape, canola, soybean, corn (maize), cotton, sugarcane, alfalfa, sorghum, and wheat.

The invention may be used to improve any one or several agronomic, horticultural, and quality traits in transgenic crop plants including, but not limited to, seed quality, seed yield, total yield, total biomass, nutritional value, protein and/or amino acid content, oil content, silage quality, feed quality, digestibility, disease and insect resistance, and cold, heat and drought tolerance.

The effect of the genetic modification on a plant can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, protein content, content of one or more amino acids, transpiration rates, general plant and/or crop yield, flowering, reproduction, seed setting, root growth, respiration rates, photosynthesis rates, metabolite composition, and the like.

In one embodiment, the present invention relates to the modification of the protein content in plant seed, resulting in seed or grains with increased digestibility/nutrient availability, increased nutrient value, increased response to feed processing, improved silage quality, improved grain quality, increased efficiency of wet or dry milling, and decreased allergenicity and/or toxicity.

In a further embodiment, by targeting the protein of interest to protein bodies, the present invention can be used to provide seed or grain with improved nutrient composition and value such as having elevated protein content or improved amino acid composition. In one embodiment, amino acid composition is improved by expressing a protein of interest that has been engineered to be enriched for one or more amino acids. These amino acids include, but are not limited to, lysine, methionine, phenylalanine, tryptophan, valine, leucine, isoleucine and threonine. Proteins engineered for improved amino acid content are described, for example, in U.S. Pat. No. 7,297,847. In another embodiment, the plants, seed, or grain of the invention are used for production of human food, animal or livestock feed, as raw material in industry, pet foods, and food products. Such products can provide increased nutrition because of the increased nutrient value. In a further embodiment, the present invention also relates to animal feed which is formulated for a specific animal type, for example, as in U.S. Pat. No. 6,774,288, which is hereby incorporated by reference in its entirety.

The seed or grain with elevated protein content may be seed or grain from any crop species including a high protein maize, for example, as in U.S. Pat. No. 6,774,288, which is hereby incorporated by reference in its entirety. High protein maize is also used for feeding non ruminant animals, such as swine, poultry, cats, dogs, horses, sheep and the like.

The invention also relates to a method for producing a protein of interest, the method comprising:
(a) growing or culturing a plant cell, plant tissue, plant or part thereof which comprise an expression cassette comprising i) a nucleic acid molecule encoding a modified protein body tag; ii) at least one nucleic acid molecule encoding a protein of interest; and iii) at least one regulatory sequence for expressing the at least one nucleic acid molecule in a plant cell; or transgenic cells, cell cultures, parts, tissues, organs or propagation material derived therefrom under conditions that provide for expression of the protein of interest; and optionally
(b) isolating the desired protein of interest.

In a further embodiment, the invention relates to a method for producing a protein of interest, the method comprising:
a) providing a plant cell comprising an expression cassette comprising
   i) a nucleic acid molecule encoding a modified protein body tag;
   ii) at least one nucleic acid molecule encoding a protein of interest; and
   iii) at least one regulatory sequence for expressing the at least one nucleic acid molecule in a plant cell; and
b) isolating the protein of interest from the plant cell.

In one embodiment, the plant cell is derived from a host cell system. In another embodiment, the plant cell is derived from a transgenic plant. The protein of interest may be isolated and/or purified from the plant cell, plant tissue, plant or part thereof according to methods known in the art, such as those provided in US Application No. 2006/0123509; Azzoni et al., 2002, Biotechnol. Bioeng., 80, 268-276; and U.S. Pat. No. 7,045,354.

The invention further relates to a method for the production of a foodstuff, feedstuff, seed, pharmaceutical, or protein of interest, the method comprising
(a) growing or culturing a plant cell, plant tissue, plant or part thereof which comprises an expression cassette comprising i) a nucleic acid molecule encoding a modified protein body tag; ii) at least one nucleic acid molecule encoding a protein of interest; and iii) at least one regulatory sequence for expressing the at least one nucleic acid molecule in a plant cell; or transgenic cells, cell cultures, parts, tissues, organs or propagation material derived therefrom; and
(b) producing and/or isolating the desired foodstuff, feedstuff, seed, pharmaceutical, or protein of interest from the plant cell, plant tissue, plant or part thereof or transgenic cells, cell cultures, parts, tissues, organs or propagation material derived therefrom.

The invention is further illustrated by the following examples, which are not to be construed in any way as imposing limitations upon the scope thereof.

### EXAMPLES

### Example 1

### Identification of Storage Protein Sequences for Protein Body Assembly

Storage proteins suitable for the invention or from which protein body tags may be derived include, but are not limited to: 16 kDa γ-zein (SEQ ID NO: 41 and SEQ ID NO: 42), 27 kDa γ-zein (SEQ ID NO: 38), 50 kDa γ-zein (SEQ ID NO: 40), cowpea glutelin-2 (SEQ ID NO: 43), and γ-kafirin proteins (for example, SEQ ID NO: 39).

Storage protein sequences for assembly of modified protein body tags in accordance with the invention can be identified by homology searches of sequence databases with known storage protein sequences. For example, a partial sequence of the Glutelin 2 protein from cowpea (Vigna unguiculata) (NCBI Accession No. AAD34914) was identified by a Position Specific Iterated BLAST (PSI-BLAST) search with the 27 kDa maize γ-zein protein. Storage protein sequences can also be identified by searching the scientific literature for previously characterized prolamins, seed storage proteins and/or other storage proteins. The γ-kafirin gene from sorghum (Sorghum bicolor) (NCBI Accession No. ADD98900) was identified by this method.

### Example 2

### Modifications of Protein Body Tags

Protein body tags modified from the corresponding wild-type protein body tag were constructed to comprise at least four domains: a signal peptide, a spacer, a repeat domain comprising one or more repeat units, and a Pro-X domain. Certain preferred modifications were done by substituting one or more of the domains with a corresponding domain from another storage protein from the same species or from a different species and/or by modifying the number of repeat units in the repeat domain.

Examples of modified protein body tags are described in Table 2 and are comprised of combinations of signal peptides, spacers, repeat domains, and Pro-X domains. Examples of domains from which modified protein body tags were derived are presented in Table 1. One or more of the domains of the modified protein body tags was derived from γ-zein proteins and/or from polypeptides homologous or orthologous to γ-zein such as 16 kDa maize γ-zeins, 27 kDa maize γ-zein, 50 kDa maize γ-zein, γ-kafirin, or Cowpea γ-zein ortholog. Each modified protein body tag shown in Table 2 comprises at least one signal peptide, a spacer, a repeat domain comprising one or more repeat units, and a Pro-X domain. To express these modified protein body tags, the corresponding nucleic acids encoding the domains were fused in a proper reading frame for expression.

TABLE 2
Examples of modifications to domains of
protein body tags. The name, source, and composition
of modified protein body tags are shown.

| | SEQ | | | Repeat | Pro-X |
|---|---|---|---|---|---|
| Name | ID NO | Signal peptide | Spacer | domain | domain |
| PBT-1 | 14 | 50 kDa γ-zein | 27 kDa γ- | 27 kDa γ- | 27 kDa γ- |
| | | | zein | zein; 1 | zein |
| | | | | repeat | |
| PBT-2 | 15 | 50 kDa γ-zein | 27 kDa γ- | 27 kDa γ- | 27 kDa γ- |
| | | | zein | zein; 2 | zein |
| | | | | repeats | |
| PBT-3 | 16 | 50 kDa γ-zein | 27 kDa γ- | 27 kDa γ- | 27 kDa γ- |
| | | | zein | zein; 3 | zein |
| | | | | repeats | |
| PBT-4 | 17 | basic | 27 kDa γ- | 27 kDa γ- | 27 kDa γ- |
| | | endochitinase b | zein | zein; 3 | zein |
| | | | | repeats | |
| PBT-5 | 18 | 16 kDa γ-zein | 27 kDa γ- | Cowpea | 27 kDa γ- |
| | | (AAL16978) | zein | γ-zein | zein |
| | | | | ortholog | |
| PBT-6 | 19 | 16 kDa γ-zein | 27 kDa γ- | Cowpea | 27 kDa γ- |
| | | (ABD63259) | zein | γ-zein | zein |
| | | | | ortholog | |
| PBT-7 | 20 | 50 kDa γ-zein | 27 kDa γ- | Cowpea | 27 kDa γ- |
| | | | zein | γ-zein | zein |
| | | | | ortholog | |
| PBT-8 | 21 | 27 kDa γ-zein | 27 kDa γ- | Cowpea | 27 kDa γ- |
| | | | zein | γ-zein | zein |
| | | | | ortholog | |
| PBT-9 | 22 | γ-kafirin | γ-kafirin | γ-kafirin; | γ-kafirin |
| | | | | 2 repeats | |
| PBT-10 | 23 | γ-kafirin | γ-kafirin | γ-kafirin; | γ-kafirin |
| | | | | 1 repeat | |
| PBT-11 | 24 | 16 kDa γ-zein | γ-kafirin | γ-kafirin; | γ-kafirin |
| | | (AAL16978) | | 1 repeat | |
| PBT-12 | 25 | 50 kDa γ-zein | γ-kafirin | γ-kafirin; | γ-kafirin |
| | | | | 1 repeat | |
| PBT-13 | 26 | 27 kDa γ-zein | γ-kafirin | γ-kafirin; | γ-kafirin |
| | | | | 1 repeat | |
| PBT-14 | 27 | 16 kDa γ-zein | γ-kafirin | γ-kafirin; | γ-kafirin |
| | | (AAL16978) | | 2 repeats | |
| PBT-15 | 28 | 50 kDa γ-zein | γ-kafirin | γ-kafirin; | γ-kafirin |
| | | | | 2 repeats | |
| PBT-16 | 29 | 27 kDa γ-zein | γ-kafirin | γ-kafirin; | γ-kafirin |
| | | | | 2 repeats | |
| PBT-17 | 30 | 16 kDa γ-zein | γ-kafirin | Cowpea | γ-kafirin |
| | | (AAL16978) | | γ-zein | |
| | | | | ortholog | |
| PBT-18 | 31 | 16 kDa γ-zein | γ-kafirin | Cowpea | γ-kafirin |
| | | (ABD63259) | | γ-zein | |
| | | | | ortholog | |
| PBT-19 | 32 | 50 kDa γ-zein | γ-kafirin | Cowpea | γ-kafirin |
| | | | | γ-zein | |
| | | | | ortholog | |
| PBT-20 | 33 | 27 kDa γ-zein | γ-kafirin | Cowpea | γ-kafirin |
| | | | | γ-zein | |
| | | | | ortholog | |
| PBT-21 | 34 | 50 kDa γ-zein | 27 kDa γ- | 27 kDa γ- | γ-kafirin |
| | | | zein | zein; | |
| | | | | 3 repeats | |
| PBT-22 | 35 | 16 kDa γ-zein | 27 kDa γ- | 27 kDa γ- | γ-kafirin |
| | | (AAL16978) | zein | zein; | |
| | | | | 3 repeats | |
| PBT-23 | 36 | 16 kDa γ-zein | 27 kDa γ- | 27 kDa γ- | γ-kafirin |
| | | (ABD63259) | zein | zein; | |
| | | | | 3 repeats | |
| PBT-24 | 45 | 27 kDa γ-zein | 27 kDa γ- | 27 kDa γ- | γ-kafirin |
| | | | zein | zein; | |
| | | | | 3 repeats | |
| PBT-25 | 46 | 50 kDa γ-zein | γ-kafirin | 27 kDa γ- | 27 kDa γ- |
| | | | | zein; | zein |
| | | | | 3 repeats | |
| PBT-26 | 47 | 50 kDa γ-zein | 27 kDa γ- | γ-kafirin; | γ-kafirin |
| | | | zein | 2 repeats | |
| PBT-27 | 48 | 16 kDa γ-zein | 27 kDa γ- | γ-kafirin; | γ-kafirin |
| | | (AAL16978) | zein | 2 repeats | |
| PBT-28 | 49 | 16 kDa γ-zein | 27 kDa γ- | γ-kafirin; | γ-kafirin |
| | | (ABD63259) | zein | 2 repeats | |
| PBT-29 | 50 | 27 kDa γ-zein | 27 kDa γ- | γ-kafirin; | γ-kafirin |
| | | | zein | 2 repeats | |
| PBT-30 | 51 | 50 kDa γ-zein | γ-kafirin | γ-kafirin; | 27 kDa γ- |
| | | | | 2 repeats | zein |
| PBT-31 | 52 | 50 kDa γ-zein | 27 kDa γ- | Cowpea | γ-kafirin |
| | | | zein | γ-zein | |
| | | | | ortholog | |
| PBT-32 | 53 | 16 kDa γ-zein | 27 kDa γ- | Cowpea | γ-kafirin |
| | | (AAL16978) | zein | γ-zein | |
| | | | | ortholog | |
| PBT-33 | 54 | 16 kDa γ-zein | 27 kDa γ- | Cowpea | γ-kafirin |
| | | (ABD63259) | zein | γ-zein | |
| | | | | ortholog | |
| PBT-34 | 55 | 27 kDa γ-zein | 27 kDa γ- | Cowpea | γ-kafirin |
| | | | zein | γ-zein | |
| | | | | ortholog | |

### Example 3

### Bioinformatics Analysis of Protein Body Tags for Allergenicity

Homologs or orthologs of the 27 kDa maize γ-zein protein were used to design modified protein body tags having no or few areas of homology with sequences from databases of allergenic proteins, which areas of homology signify potential allergenicity. The modified protein body tags designed were tested for their potential allergenic cross-reactivity.

A total of 41 sequences were subject of the analysis including the 34 modified protein tags as depicted in Table 2 as well as the following wild-type sequences: the 27 kDa γ-zein from Zea mays (AAL16977), the 50 kDa γ-zein from Zea mays (AAL16979), the 16 kDa γ-zein from Zea mays (AAL16978), the 16 kDa γ-zein mucronate mutant from Zea mays (ABD63259), the γ-kafirin from Sorghum bicolor (ADD98900.1), the Glutelin 2 from Vigna unguiculata (AAD34914), and the Zera tag without its signal peptide (Llop-Tous et al., 2010, J. Biol. Chem. 285 (46): 35633-44).

### Assessment of Allergenic Cross-Reactivity

IgE cross-reactivity between a protein and a known allergen is considered a possibility when there is more than 35% shared identity over a segment of 80 or greater amino acids. This homology is considered the potential allergen threshold as presently recommended by the Codex Alimentarius Commission. (Codex Alimentarius Commission, Appendix III, Guideline for the conduct of food safety assessment of foods derived from recombinant-DNA plants, and Appendix IV, Annex on the assessment of possible allergenicity, Joint FAO/WHO Food Standard Programme, Twenty-Fifth Session, Rome, Italy, 30 Jun.-5 Jul. 2003, pp. 47-60. Codex Alimentarius Commission, Foods derived from modern biotechnology, FAO/WHO, Rome, 2009, pp. 1-85).

### The 80 Amino Acid Test:

The amino acid sequences for the protein body tags depicted in Table 2 and for the wild-type sequences tested were subdivided into all possible overlapping 80-amino acid segments. Each of these 80-amino acid segments was compared in silico to all proteins in the FARRP Allergen Protein Database via a protein-protein FASTA (version 34.26.5; Apr. 26, 2007) analysis. The default parameters of the FASTA program were used, including the default substitution scoring matrix of BLOSUM 50, with one exception: the threshold score for optimization was set to 20.

Since the total protein sequence was analyzed incrementally in 80-amino acid segments, the query length for each of the analyses was 80 amino acids. Thus, the percent identity for a given alignment was determined by dividing the number of identical amino acids within the alignment by 80. In instances where gaps were inserted into the query sequence to achieve the optimal alignment, percent identity was calculated by dividing the number of identical amino acid residues in the alignment by the alignment length of overlap if the overlap length was greater than 80. A query protein which showed greater than 35% shared identity over >80 amino acids criteria (Klinglmayr et al., 2009, Allergy 64:647-651) to a known or putative allergen would be identified as potentially requiring additional studies, on a case-by-case basis, to determine the likelihood of the protein being allergenic.

The Food Allergy Research and Resource Program (FARRP) Allergen Protein Database (version 10.00; release date January 2010; allergenonline.com) containing 1471 entries was utilized for the bioinformatics assessments of potential allergenicity. These 1471 entries are comprised of known or putative food, respiratory, venom/salivary, or contact allergenic proteins. All allergen database entries have been vetted by a panel of seven academic allergy experts based on published evidence of allergenicity.

### The 8 Amino Acid Test:

The amino acid sequences for the protein body tags depicted in Table 2 and for the wild-type sequences tested were sequences were additionally submitted to an analysis using a custom comparison (word-match) program which provides an exhaustive search of all possible eight-amino acid subsegments of the query protein against all possible eight-amino acid segments in proteins in the FARRP Allergen Protein Database. Regions of at least eight consecutive amino acids which are identical between a submitted protein and a known allergen will be identified by this search.

This eight-amino acid search was originally suggested based on the concept that eight or more amino acids is a representative minimal size for an IgE-binding epitope (Metcalfe et al., 1996, Crit. Rev. Food Sci. Nutr. 36:S165-S186). Bannon and Ogawa (2006) compiled a list of characterized linear IgE-binding epitopes from major allergens and, although one epitope from a wheat Ω-5 gliadin was only four amino acids long, the majority of characterized epitopes were indeed eight amino acids or longer. (Bannon and Ogawa, 2006, Mol. Nutr. Food Res. 50:638-644). However, this search does not detect conformational epitopes which are formed when non-linear amino acids are brought together by the higher-order folding of the protein. Moreover, the utility of such an eight-amino acid analysis has been questioned due to the high rate of false positives identified by this search (Silvanovich et al., 2006, Toxicol. Sci. 90:252-258; Hileman et al., 2002, Int. Arch. Allergy Immunol. 128:280-291).

### Results

Results from the 80 Amino Acid (80 AA) and 8 Amino Acid (8AA) tests are presented in Table 3. For the 80 AA test, the assessment identifies all the wild-type sequences (i.e. the 27 kDa γ-zein from Zea mays (AAL16977), the 50 kDa γ-zein from Zea mays (AAL16979), the 16 kDa γ-zein from Zea mays (AAL16978), the 16 kDa γ-zein mucronate mutant from Zea mays (ABD63259), the γ-kafirin from Sorghum bicolor (ADD98900.1), the Glutelin 2 from Vigna unguiculata (AAD34914), and the Zera tag without its signal peptide as containing regions with 35% or higher shared identity over a segment of 80 or greater amino acids to known allergens. This surpasses the threshold level dictated by Codex Alimentarius Commission (2003, 2009).

The 8 AA test identifies that 27 kDa γ-zein (AAL16977), γ-kafirin from Sorghum bicolor (ADD98900.1), and the 50 kDa γ-zein (AAL16979) to contain 8 amino acid regions with 8 or greater peptides classified as allergens.

The Zera protein tag contains regions with 35% shared identity to known allergens. In contrast, all 34 modified protein body tags as depicted in Table 2 do not contain any regions with 35% or higher shared identity nor regions with 8 or greater peptides classified as allergens. Additionally, PBT-1, PBT-3, PBT-8, and PBT-9 modified by omitting the spacer also did not contain any regions with 35% or higher shared identity nor regions with 8 or greater peptides classified as allergens.

TABLE 3
Summary of 80-amino acid (AA) regions and 8-amino acid
regions of query sequences with similarity to known allergens.

| | 80 AA Test | |
|---|---|---|
| | (Number of | 8AA Test |
| | Regions | (number of |
| Name | ≧35%) | regions) |
| PBT-1 (SEQ ID NO: 14) | 0 | 0 |
| PBT-1 without spacer (SEQ ID NO: 57) | 0 | 0 |
| PBT-2 (SEQ ID NO: 15) | 0 | 0 |
| PBT-3 (SEQ ID NO: 16) | 0 | 0 |
| PBT-3 without spacer (SEQ ID NO: 59) | 0 | 0 |
| PBT-4 (SEQ ID NO: 17) | 0 | 0 |
| PBT-5 (SEQ ID NO: 18) | 0 | 0 |
| PBT-6 (SEQ ID NO: 19) | 0 | 0 |
| PBT-7 (SEQ ID NO: 20) | 0 | 0 |
| PBT-8 (SEQ ID NO: 21) | 0 | 0 |
| PBT-8 without spacer (SEQ ID NO: 61) | 0 | 0 |
| PBT-9 (SEQ ID NO: 22) | 0 | 0 |
| PBT-9 without spacer (SEQ ID NO: 63) | 0 | 0 |
| PBT-10 (SEQ ID NO: 23) | 0 | 0 |
| PBT-11 (SEQ ID NO: 24) | 0 | 0 |
| PBT-12 (SEQ ID NO: 25) | 0 | 0 |
| PBT-13 (SEQ ID NO: 26) | 0 | 0 |
| PBT-14 (SEQ ID NO: 27) | 0 | 0 |
| PBT-15 (SEQ ID NO: 28) | 0 | 0 |
| PBT-16 (SEQ ID NO: 29) | 0 | 0 |
| PBT-17 (SEQ ID NO: 30) | 0 | 0 |
| PBT-18 (SEQ ID NO: 31) | 0 | 0 |
| PBT-19 (SEQ ID NO: 32) | 0 | 0 |
| PBT-20 (SEQ ID NO: 33) | 0 | 0 |
| PBT-21 (SEQ ID NO: 34) | 0 | 0 |
| PBT-22 (SEQ ID NO: 35) | 0 | 0 |
| PBT-23 (SEQ ID NO: 36) | 0 | 0 |
| PBT-24 (SEQ ID NO: 45) | 0 | 0 |
| PBT-25 (SEQ ID NO: 46) | 0 | 0 |
| PBT-26 (SEQ ID NO: 47) | 0 | 0 |
| PBT-27 (SEQ ID NO: 48) | 0 | 0 |
| PBT-28 (SEQ ID NO: 49) | 0 | 0 |
| PBT-29 (SEQ ID NO: 50) | 0 | 0 |
| PBT-30 (SEQ ID NO: 51) | 0 | 0 |
| PBT-31 (SEQ ID NO: 52) | 0 | 0 |
| PBT-32 (SEQ ID NO: 53) | 0 | 0 |
| PBT-33 (SEQ ID NO: 54) | 0 | 0 |
| PBT-34 (SEQ ID NO: 55) | 0 | 0 |
| AAL16977_27 kD_gamma_zein_[Zea_mays] | 1550 | 6 |
| (SEQ ID NO: 38) | | |
| ADD98900.1_gamma_kafirin_[Sorghum_bicolor] | 827 | 6 |
| (SEQ ID NO: 39) | | |
| AAL16979_50 kD_gamma_zein_[Zea_mays] | 6738 | 28 |
| (SEQ ID NO: 40) | | |
| AAL16978_16 kD_gamma_zein_[Zea_mays] | 731 | 0 |
| (SEQ ID NO: 41) | | |
| ABD63259_16_kDa_gamma_zein_mucronate_mutant_[Zea_mays] | 438 | 0 |
| (SEQ ID NO: 42) | | |
| AAD34914_Glutelin_2_[Vigna_unguiculata] | 0 | 0 |
| (SEQ ID NO: 43) | | |
| Zera Tag without signal peptide | 72 | 0 |
| (part of SEQ ID NO: 37) | | |

The results from the 80 AA test corresponds to the number of 80 amino acid regions with 35% or greater sequence identity to an 80-AA region of a known allergen.
The results from the 8 AA test corresponds to the number of 8 amino acid regions with 8 or greater peptides classified as allergen.

Some of the allergen regions identified in the 80 AA test for the wild-type sequences are as follows. The 27 kDa γ-zein (AAL16977) showed 38 to 41% identity to known allergens grouped under Triticum Alpha/beta gliadin IgE & celiac and Triticum gamma gliadin IgE & celiac. The 50 kDa γ-zein (AAL16979) showed 39 to 56% identity to allergens grouped under Triticum HMW glutenin and Triticum omega-5 gliadin Tri a 19. The γ-kafirin (ADD98900.1) showed 40 to 41% identity to allergens grouped under Triticum gamma gliadin IgE & celiac. The Zera tag without its signal peptide showed 35% identity to allergens grouped under Triticum Alpha/beta gliadin IgE & celiac and under Triticum omega-5 gliadin Tri a 19.

### Example 4

### Construction of Expression Cassettes

General cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA are carried out as described in Sambrook et al. (1989, Cold Spring Harbor Laboratory Press: ISBN 0-87969-309-6) or Kaiser, Michaelis and Mitchell (1994, “Methods in Yeast Genetics,” Cold Spring Harbor Laboratory Press: ISBN 0-87969-451-3).

Protein body tags that passed the allergen screen were fused to the native C-terminal 27 kDa γ-zein sequence or to another protein-of-interest which may include a reporter gene. Non-limiting examples of proteins of interest may also include green fluorescent protein (GFP), DsRED, epidermal growth factor (EGF), or a quality plant protein. Fusion proteins for expression in cell culture may also further comprise a histidine tag or other detectable marker. Histidine tags and other detectable markers are described, for example, in Hochuli et al., 1988, Bio/Technology: 1321-1325; Watson, et al., 2004, Program No. 73.8, Sigma-Aldrich Co., Page 1; Smith et al., 1988, Gene 67:31-40; Pharmacia LKB Biotechnology, 1991, Analects, 19(3):1-8; Brizzard et al., 1994, BioTechniques 16(4): 730-734; Watson et al., 2005, FASEB/ASBMB Experimental Biology, Poster No. 213.6, Sigma-Aldrich Co., Page 1; Zheng et al., 1997, Gene 186: 55-60; Sato et al., 1997, Biotechniques 23 (2): 254-256; and Sano et al., 1992, Proceedings of the National Academy of Sciences USA 89: 1534-1538.

The fusion proteins containing the protein body tag and one or more protein of interest were generated through reverse translation of the protein sequence, codon optimization of the resulting nucleotide sequence, and DNA synthesis. DNA synthesis was performed by a range of commercial vendors including Epoch Life Science (Missouri City, Tex.), Blue Heron Biotechnology (Bothell, Wash.) and DNA 2.0 (Menlo Park, Calif.). After synthesis, the DNA encoding the fusion protein was cloned into standard cloning vectors, such as pUC-type vectors, and sequenced. The expression cassette was assembled in a cloning vector by cloning the synthesized DNA encoding the fusion protein downstream of a plant promoter and optionally upstream of a terminator region.

### Example 5

### Construction of Plant Transformation Vectors

Plant transformation binary vectors such as pBi-nAR are used (Höfgen & Willmitzer 1990, Plant Sci. 66:221-230). Construction of the binary vectors was performed by ligation of the expression cassette into the binary vector. Further examples for plant binary vectors are the pSUN300 or pSUN2-GW vectors. These binary vectors contain an antibiotic resistance gene driven under the control of the NOS promoter. Expression cassettes were cloned into the multiple cloning site of the pEntry vector using standard cloning procedures. pEntry vectors are combined with a pSUN destination vector to form a binary vector by the use of the GATEWAY technology (Invitrogen, webpage at invitrogen.com) following the manufacturer's instructions. The recombinant vector containing the expression cassette was transformed into Top10 cells (Invitrogen) using standard conditions. Transformed cells were selected on LB agar containing 50 μg/ml kanamycin grown overnight at 37° C. Plasmid DNA was extracted using the QiAprep Spin Miniprep Kit (Qiagen) following manufacturer's instructions. Analysis of subsequent clones and restriction mapping was performed according to standard molecular biology techniques (Sambrook et al., 1989, Molecular Cloning, A Laboratory Manual. 2nd Edition. Cold Spring Harbor Laboratory Press. Cold Spring Harbor, N.Y.).

### Example 6

### Agrobacterium-Mediated Plant Transformation

Agrobacterium-mediated plant transformation was performed using standard transformation and regeneration techniques (Gelvin et al., Plant Molecular Biology Manual, 2nd ed. Kluwer Academic Publ., Dordrecht 1995 in Sect., Ringbuc Zentrale Signatur:BT11-P; Glick et al. Methods in Plant Molecular Biology and Biotechnology, S. 360, CRC Press, Boca Raton, 1993). For example, Agrobacterium-mediated transformation can be performed using the GV3 (pMP90) (Koncz et al., 1986, Mol. Gen. Genet. 204:383-396) or LBA4404 (Clontech) Agrobacterium tumefaciens strain. Agrobacterium cells containing the transformation vector were used to transform plant cells for generation of plant cell cultures. Examples of maize cell culture transformation and immature maize embryo transformation are shown below.

### Maize Cell Culture Transformation

Black Mexican Sweetcorn (BMS) cells were transformed with Agrobacterium tumefaciens strain LBA4404(pSB1) containing one of various transformation vectors. The transformation vectors carry a cassette comprising the chimeric gene of interest described above driven by a strong constitutive promoter. This cassette was flanked on either side by reporter cassettes for ease of identifying transformed tissue. The protocol for transformation of BMS cells is shown below:

### BMS Cell Maintenance:

Cells were subcultured onto fresh M-MS-715 solid medium (Table 4) every month and kept at 27° C.

### Agrobacterium Preparation:

Agrobacterium cells were grown on solid YP medium with antibiotic(s) for 1-2 days. Two loops of Agrobacterium cells were collected and suspended in 2 mls M-LS-002 medium (LS-inf) to make a 1.0 OD Agrobacterium suspension, which was kept on a shaker for 10 min-2 hrs at 1,200 rpm prior to exposure to BMS cells.

### Inoculation and Co-Cultivation:

Approximately 100 mg of white and friable BMS cells were transferred into the tube containing Agrobacterium cells in LS-inf solution (M-LS-002, Table 5). Agrobacterium infection was carried out by inverting the tube several times over the course of 30 minutes. The mixture was poured onto the surface of 2 layers of filter paper in an empty plate. The first layer of filter paper with cells was transferred onto the co-cultivation medium (M-LS-011, Table 6). The infected cells were cultured in the dark at 22° C. for 2-4 days.

### Recovery:

Following co-culture, the cells on filter paper were placed onto recovery media (M-LS-719, Table 7) for 5-7 days in the dark at 27° C.

Selection 1:

Following recovery, the cells on filter paper were transferred to selection media (M-MS-715+150 mg Timentin+0.75 μM Pursuit) and were grown for 1 week in the dark at 27° C.

Selection 2:

Cells were then transferred from the filter paper to the same medium by sections to select for transformed cells. Cultures were placed on selection media (M-LS-715+150 mg Timentin+0.75 μm in 27° C. incubator under cool-white light (100 μE·m-2.s-1) and allowed to grow for 10 days. Transformed, growing calli were bulked in culture and then subjected to homogenization and density centrifugation as described by Torrent et al., 2009, to isolate and assess protein body formation, protein accumulation, and in vitro digestibility.

TABLE 4
M-MS-715 medium.

| M-MS-715 | | |
|---|---|---|
| Ingredients | Final Amt | |
| MS salts | 4.30 | g/L |
| Sucrose | 30 | g/L |
| Proline | 1.16 | g/L |
| Casamino acid | 1 | g/L |
| L-Asparagine | 150 | mg/L |
| monohydrate | | |
| Nicotinic acid | 0.5 | mg/L |
| Pyridoxine HCl | 0.5 | mg/L |
| Thiamine HCl | 1 | mg/L |
| Myo-inositol | 100 | mg/L |
| MES | 500 | mg/L |
| Purified Agar | 8 | g/L |
| 2,4-D | 1 | mg/L |

TABLE 5
M-LS-002 medium.

| M-LS-002 | | |
|---|---|---|
| Ingredients | Final Amt | |
| MS salts | 4.3 | g/L |
| Glucose | 36 | g/L |
| Sucrose | 68.5 | g/L |
| 2,4-D | 1.5 | mg/L |
| Nicotinic acid | 0.5 | mg/L |
| Pyridoxine HCl | 0.5 | mg/L |
| Thiamine HCl | 1 | mg/L |
| Myo-inositol | 100 | mg/L |
| Casamino acid | 1 | g/L |
| Acetosyringone | 200 | μM |

TABLE 6
M-LS-011 medium.

| M-LS-011 | | |
|---|---|---|
| Ingredients | Final Amt | |
| MS salts | 4.3 | g/L |
| Glucose | 10 | g/L |
| Sucrose | 20 | g/L |
| 2,4-D | 1.5 | mg/L |
| Nicotinic acid | 0.5 | mg/L |
| Pyridoxine HCl | 0.5 | mg/L |
| Thiamine HCl | 1 | mg/L |
| Myo-inositol | 100 | mg/L |
| L-proline | 700 | mg/L |
| MES | 500 | mg/L |
| Purified Agar | 8 | g/L |
| AgNO₃ | 15 | μM |
| Acetosyringone | 200 | μM |

TABLE 7
M-MS-719 medium.

| M-MS-719 | | |
|---|---|---|
| Ingredients | Final Amt | |
| MS salts | 4.30 | g/L |
| Sucrose | 30 | g/L |
| Proline | 1.16 | g/L |
| Casamino acid | 1 | g/L |
| L-Asparagine | 150 | mg/L |
| monohydrate | | |
| Nicotinic acid | 0.5 | mg/L |
| Pyridoxine HCl | 0.5 | mg/L |
| Thiamine HCl | 1 | mg/L |
| Myo-inositol | 100 | mg/L |
| 2,4-D | 1 | mg/L |
| MES | 500 | mg/L |
| Purified Agar | 8 | g/L |
| Silver Nitrate | 15 | μM |
| Timentin | 150 | mg/L |

### Immature Maize Embryo Transformation

Immature embryos were transformed according to the procedure outlined in Peng et al., 2006 (WO2006/136596) which is incorporated herein by reference in its entirety. Modifications that encourage growth of somatic embryogenic callus rather than organogenic callus were employed. These changes included use of smaller immature embryos (−1 mm), maize lines more likely to produce type II callus such as the F1 hybrid, J553xHiIIA, and wrapping culture plates in parafilm instead of micropore tape. After approximately 1 month on selection media, transgenic calli of sufficient embryogenic morphology were bulked and analyzed for protein body formation.

### Example 8

### Analysis of Protein Body Formation in Cell Cultures

### Isolation of Protein Bodies

One gram of callus was homogenized in 2 ml buffer containing 100 mM Tris HCl, pH 8.0, 50 mM KCl, 6 mM MgCl₂, 1 mM EDTA, 0.4 M NaCl and protease inhibitors. The homogenate was filtered through 2 layers of miracloth to remove the debris. The filtrate was centrifuged at 50×g for 5 min. at 4° C. The resulting supernatant was loaded onto a multi-step 20/30/42/56 percent sucrose gradient buffered with the buffer mentioned above. The gradient was centrifuged at 4° C. for 2 hrs at 80,000×g by using a swinging bucket rotor (SW28). The interphases were collected as well as the pellet and protein fractions were analyzed using SDS-PAGE. Western blot analyses were performed in order to detect and estimate the amount of protein. Proteins comprising a histidine tag or other detectable marker were detected using anti tag antibodies. Methods for detecting tagged proteins are known in the art and are provided for example in Hochuli et al., 1988, Bio/Technology: 1321-1325; Watson, et al., 2004, Program No. 73.8, Sigma-Aldrich Co., Page 1; Smith et al., 1988, Gene 67:31-40; Pharmacia LKB Biotechnology, 1991, Analects, 19(3):1-8; Brizzard et al., 1994, BioTechniques 16(4): 730-734; Watson et al., 2005, FASEB/ASBMB Experimental Biology, Poster No. 213.6, Sigma-Aldrich Co., Page 1; Zheng et al., 1997, Gene 186: 55-60; Sato et al., 1997, Biotechniques 23 (2): 254-256; and Sano et al., 1992, Proceedings of the National Academy of Sciences USA 89: 1534-1538.

Digestibility studies are performed on the isolated protein bodies by treating with protease cocktail as described below.

### Proteolytic Digestions

Prior to trypsin digestion, isolated protein bodies are resuspended in phosphate buffered saline (PBS, 6 mM sodium phosphate, pH 7.4, 1 mM potassium phosphate, 153 mM sodium chloride) at a protein concentration of approx. 5 mg/ml. Trypsin sensitivity of the protein is determined for proteins isolated from transgenic samples and corresponding wild type extracts. A 0 minute control is prepared by removing an aliquot of sample and heating at 95° C. for 5 minutes with 3× loading buffer (30% glycerol, 6% sodium dodecyl sulfate, 75 mM DTT, 187.5 mM Tris, 0.015% bromophenol blue, pH 6.8). Trypsin is added into a bulk reaction to a final concentration of 1150 Units/ml and the reaction mixture is incubated at 37° C. Aliquots are removed from the incubating reaction mixes after incubation for 1, 5 and 60 minutes and the reaction is stopped by heating the aliquots at 95° C. for 5 minutes with 3× loading buffer. A 60 minute control without trypsin is prepared by incubating the aliquot of extract at 37° C. for 60 minutes in the absence of trypsin and stopping the reaction by the addition of 3× loading buffer followed by heating for 5 minutes at 95° C.

Prior to pepsin digestion, protein is resuspended in 1×G-con (0.84 N HCl, pH 1.2, 35 mM sodium chloride) at a protein concentration of approx. 10 mg/ml. Pepsin sensitivity of the expressed protein is determined for transgenic and wild type extracts. A 0 minute control is prepared by removing an aliquot of sample, adding Tris base, and heating at 95° C. for 5 minutes with 3× loading buffer. Pepsin is added to a bulk reaction to a final concentration of 5 Units/μg protein and the reaction mixture was incubated at 37° C. Aliquots are removed from the incubating reaction mixes after incubation for 1, 5 and 60 minutes and the reaction is stopped by neutralization with Tris base pH ˜11 and heating the aliquots at 95° C. for 5 minutes with 3× loading buffer. A 60 minute control without pepsin is prepared by incubating the aliquot of extract at 37° C. for 60 minutes in the absence of pepsin and stopping the reaction with Tris and the addition of 3× loading buffer followed by heating for 5 minutes at 95° C. Separation of proteins by SDS-polyacrylamide gel electrophoresis (PAGE) and detection of expressed protein by western blotting is described below.

### SDS-PAGE and Western Blot Analysis

Aliquots of the trypsin and pepsin reaction mixtures are subjected to SDS-PAGE on a 4-20% polyacrylamide gradient gel followed by electroblotting onto nitrocellulose membrane (Invitrogen; Carlsbad, Calif.). To detect remaining His tagged protein, the membrane is blocked in 5% nonfat dry milk in 25 mM Tris-HCl, 140 mM NaCl, 3 mM KCl, pH 7.4 and probed with rabbit anti-His antibody in 0.05% Tween-20, 25 mM Tris-HCl, 140 mM NaCl, 3 mM KCl, pH 7.4. Secondary antibody linked to horseradish peroxidase is used to bind to the primary antibody and is visualized by methods provided by the substrate manufacturer. Molecular weight markers are indicated on the blot.

Analysis of protein body formation is also described in Torrent et al., 2009.

### Example 9

### Production of Transgenic Plants

### Maize

Transgenic maize plant production is described, for example, in U.S. Pat. No. 5,591,616 and WO/2006136596, both of which are hereby incorporated by reference in their entirety. Transformation of maize may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of maize (Zea Mays L.) can also be performed with a modification of the method described by Ishida et al. (1996, Nature Biotech. 14:745-750). The inbred line A188 (University of Minnesota) or hybrids with A188 as a parent are good sources of donor material for transformation (Fromm et al., 1990, Biotech 8:833), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system is described in WO 94/00977 and WO 95/06722. Vectors are constructed as described. Various selection marker genes are used including the maize gene encoding a mutated acetohydroxy acid synthase (AHAS) enzyme (U.S. Pat. No. 6,025,541). Similarly, various promoters are used to regulate the trait gene to provide constitutive, developmental, inducible, tissue or environmental regulation of gene transcription.

Excised embryos are grown on callus induction medium, then maize regeneration medium, containing imidazolinone as a selection agent. The petri dishes are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Tobacco

Transgenic tobacco production is described, for example, by Torrent et al., 2009, Methods in Molecular Biology, Recombinant Proteins from Plants, 483:193-208.

### Soybean

Transformation of soybean can be performed using, for example, a technique described in European Patent No. EP 0424 047, U.S. Pat. No. 5,322,783, European Patent No. EP 0397 687, U.S. Pat. No. 5,376,543 or U.S. Pat. No. 5,169,770, or by any of a number of other transformation procedures known in the art. Soybean seeds are surface sterilized with 70% ethanol for 4 minutes at room temperature with continuous shaking, followed by 20% (v/v) bleach supplemented with 0.05% (v/v) TWEEN for 20 minutes with continuous shaking. Then the seeds are rinsed 4 times with distilled water and placed on moistened sterile filter paper in a petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile petri dish and air-dried to a moisture content less than 20% (fresh weight) in a sealed petri dish until further use.

### Wheat

A specific example of wheat transformation can be found in PCT Application No. WO 93/07256. Transformation of wheat can also be performed with the method described by Ishida et al. (1996, Nature Biotech. 14:745-750). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system is described in WO 94/00977 and WO 95/06722, which are hereby incorporated by reference in its entirety. Vectors are constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, inducible, developmental, tissue or environmental regulation of gene transcription.

After incubation with Agrobacterium, the embryos are grown on callus induction medium, then regeneration medium, containing imidazolinone as a selection agent. The petri dishes are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

Brassica napus

Canola may be transformed, for example, using methods such as those disclosed in U.S. Pat. Nos. 5,188,958; 5,463,174; 5,750,871; EP1566443; WO02/00900; and the like. For example, seeds of canola are surface sterilized with 70% ethanol for 4 minutes at room temperature with continuous shaking, followed by 20% (v/v) CLOROX supplemented with 0.05% (v/v) TWEEN for 20 minutes, at room temperature with continuous shaking. Then, the seeds are rinsed four times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use.

Agrobacterium tumefaciens culture is prepared from a single colony in LB solid medium plus appropriate antibiotics (e.g. 100 mg/l streptomycin, 50 mg/l kanamycin) followed by growth of the single colony in liquid LB medium to an optical density at 600 nm of 0.8. Then, the bacteria culture is pelleted at 7000 rpm for 7 minutes at room temperature, and resuspended in MS (Murashige et al., 1962, Physiol. Plant. 15:473-497) medium supplemented with 100 mM acetosyringone. Bacteria cultures are incubated in this pre-induction medium for 2 hours at room temperature before use. The axis of soybean zygotic seed embryos at approximately 44% moisture content are imbibed for 2 hours at room temperature with the pre-induced Agrobacterium suspension culture. (The imbibition of dry embryos with a culture of Agrobacterium is also applicable to maize embryo axes). The embryos are removed from the imbibition culture and are transferred to petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/l carbenicillin or 300 mg/l cefotaxime to kill the Agrobacteria. The liquid medium is used to moisten the sterile filter paper. The embryos are incubated during 4 weeks at 25° C., under 440 μmol m²s¹ and a 12 hour photoperiod. Once the seedlings have produced roots, they are transferred to sterile soil. The medium of the in vitro plants is washed off before transferring the plants to soil. The plants are kept under a plastic cover for 1 week to favor the acclimatization process. Then the plants are transferred to a growth room where they are incubated at 25° C., under 440 μmol m²s¹ light intensity and 12-hour photoperiod for about 80 days.

Samples of the primary transgenic plants (TO) are analyzed by PCR to confirm the presence of T-DNA. These results can be confirmed by Southern hybridization wherein DNA is electrophoresed on a 1% agarose gel and transferred to a positively charged nylon membrane (Roche Diagnostics). The PCR DIG Probe Synthesis Kit (Roche Diagnostics) is used to prepare a digoxigenin labeled probe by PCR as recommended by the manufacturer.

### Rice

Rice may be transformed using methods disclosed in U.S. Pat. Nos. 4,666,844; 5,350,688; 6,153,813; 6,333,449; 6,288,312; 6,365,807; 6,329,571, and the like.

### Example 10

### Analysis of Protein Body Formation and Protein Accumulation in Transgenic Plants and Plant Tissue

Analysis of protein body formation in transgenic plants, plant parts, plant cell cultures, or plant tissues, which includes maize cell cultures, leaves, stems, and/or seed, can be performed by the methods provided in Example 8. The ratio of plant tissue to homogenization buffer can be adjusted depending on the tissue. For example, one gram of corn kernels is homogenized in 4 ml of homogenization buffer.

Protein body formation in transgenic plants, plant parts, plant cell cultures, or plant tissues, can also be analyzed by the methods described by Torrent et al. (2009, Methods in Molecular Biology, Recombinant Proteins from Plants, Humana Press, 483:193-201). The protein body tag is fused to a fluorescent marker and expressed in the transgenic plant. Protein bodies can be observed by microscopy, for example, by mounting leaf sections of the transgenic plants in water and identifying protein bodies in epidermal cells using confocal microscopy. Protein bodies can also be observed by immunodetection. Analysis of protein body formation by fluorescence and electron microscopy is also described, for example, in Loussert et al. (2008, J. Cereal Sci. 47: 445-456).

Increased protein body formation or protein accumulation in the protein bodies can lead to increased protein content and/or content of one or more amino acids in the transgenic plant, plant part, plant cell culture, or plant tissue relative to a corresponding control plant, plant part, plant cell culture, or plant tissue. Control plants, plant parts, plant cell cultures, or plant tissues can include wild-type plant, plant part, plant cell culture, or plant tissue corresponding to the transgenic plants, plant parts, plant cell cultures, or plant tissues or transgenic plants, plant parts, plant cell cultures, or plant tissues with an expression cassette comprising an unmodified wild-type protein body tag.

Protein content and content of one or more amino acids of transgenic and corresponding wild-type plants, plant parts, plant cell cultures, or plant tissues and seeds can be evaluated by methods known in the art, for example, as described for corn in U.S. Publication Serial No. 2005/0241020 which is hereby incorporated by reference in its entirety. An example for analyzing the protein content in leaves and seeds can be found by Bradford (1976, Anal. Biochem. 72:248-254). For example, quantification of total seed protein, 15-20 seeds are homogenized in 250 μl of acetone in a 1.5-ml polypropylene test tube. Following centrifugation at 16,000 g, the supernatant is discarded and the vacuum-dried pellet is resuspended in 250 μl of extraction buffer containing 50 mM Tris-HCl, pH 8.0, 250 mM NaCl, 1 mM EDTA, and 1% (w/v) SDS. Following incubation for 2 h at 25° C., the homogenate is centrifuged at 16,000 g for 5 min and 200 ml of the supernatant will be used for protein measurements. In the assay, γ-globulin was used for calibration. For protein measurements, Lowry DC protein assay (Bio-Rad) or Bradford-assay (Bio-Rad) or bicinchoninic acid assay (Smith et al., 1985) is used. The latter method was used to quantitate protein in maize cell cultures.

Western blot analysis can be used to quantitate accumulation of recombinant protein in protein bodies isolated from plant tissues and cell culture. Plant tissue callus is homogenized in buffer containing 100 mM Tris HCl, pH 8.0, 50 mM KCl, 6 mM MgCl₂, 1 mM EDTA, 0.4 M NaCl and protease inhibitors. Samples are mixed with 3× loading buffer, denatured for 5 min at 95° C., loaded on 4-20% Tris-HCl gels (BioRad), and electrophoresed at 200V in 1× tris-glycine-SDS running buffer. Protein is transferred from the gel to nitrocellulose (iBlot gel transfer stacks, Invitrogen). Blocking is performed for 1 hr at room temperature in 5% bovine serum albumin (BSA) in 1×TBS by shaking followed by incubation in primary antibody (Anti-His antibody, mouse, GE Healthcare #27471001) with 1:3000 dilution. Blots are incubated in ECL Plex goat anti-mouse IgG, Cy3, GE Healthcare #PA43010V (1:2500) and washed 3 times for 10 min in 1×TBST followed by 3 times for 5 min in 1×TBS buffer. Blots are allowed to air dry at least 30 minutes and scanned using the Typhoon 9400 Variable Mode Imager (Amersham Biosciences) (Cy3 channel; 100-200 μm resolution). Quantitation of a standard curve and samples is done using Image Quant TL 7.0 software from GE Healthcare (1D Gel Analysis module).

An example of amino acid analysis of transgenic seed can be found for example for corn in U.S. Publication Serial No. 2005/0241020. For example, mature seed samples are ground with an IKA A11 basic analytical mill. The samples are re-ground and analyzed for complete amino acid profile (AAP) using the Association of Official Analytical Chemists (AOAC) official method 982.30 E (a, b, c), CHP 45.3.05, 2000, with four repetitions. The samples are also analyzed for crude protein (2000, Combustion Analysis (LECO) AOAC Official Method 990.03), crude fat (2000, Ether Extraction, AOAC Official Method 920.39 (A)), and moisture (2000, vacuum oven, AOAC Official Method 934.01).

### Analysis of Protein Body Formation and Recombinant Protein Accumulation in Transgenic Plant Cells.

Protein body formation was detected in BMS maize cell culture by Western blotting using anti-His tag antibodies following the method described above in Example 10. An example is provided in FIG. 7. To assess the ability of modified protein body tags to induce the formation of protein bodies, His-tagged fusions of modified protein body tags described herein with the C-terminus of SEQ ID NO: 38 (corresponding to positions 112 to 223 of the amino acid sequence of SEQ ID NO: 38) were created (see FIG. 5). Protein body tags further modified by omitting the spacer were included in the analysis. A 8×His-tagged version of SEQ ID NO: 38 was used as positive control. Constructions of expression cassettes and plant transformation vectors containing the modified protein body tag fusions were performed as described in Example 4 and 5, respectively. Agrobacterium-mediated transformation of maize tissue cultures was done according to Example 6. Transgenic calli were harvested and analyzed for protein body formation using protocols described in Example 8. Table 8 provides the results of the immunoblot analysis (see also FIG. 7 as an example). Results are indicated with (+) or (−) corresponding to presence or absence (within the limits of detection of the Western blotting technique) of protein bodies, respectively.

TABLE 8
Detection of protein body formation based on immunoblot analysis of the 8xHis-
tagged protein body tag (PBT) fusions to the C-terminus of SEQ ID NO: 38* over-expressed
in BMS maize cell cultures, and 8xHis-tagged native γ-zein (8xHis tagged SEQ ID NO: 38)
as the control.

| | PBT | PBT | | | | | |
|---|---|---|---|---|---|---|---|
| | Amino | Nucleic | | | | | |
| | Acid | Acid | | | | | |
| Includes | SEQ | SEQ | Signal | | Repeat | Pro-X | Screen- |
| PBT | ID NO | ID NO | peptide | Spacer | domain | domain | ing results |
| control | 37 | 89 | 27 kDa γ- | 27 kDa | 27 kDa γ- | 27 kDa γ- | + (control) |
| Native | | | zein | γ-zein | zein; | zein | |
| γ-zein | | | | | 8 repeat | | |
| PBT | | | | | | | |
| PBT-1 | 14 | 65 | 50 kDa γ- | 27 kDa | 27 kDa γ- | 27 kDa γ- | + |
| | | | zein | γ-zein | zein; | zein | |
| | | | | | 1 repeat | | |
| PBT-3 | 16 | 66 | 50 kDa γ- | 27 kDa | 27 kDa γ- | 27 kDa γ- | + |
| | | | zein | γ-zein | zein; | zein | |
| | | | | | 3 repeats | | |
| PBT-4 | 17 | 67 | basic | 27 kDa | 27 kDa γ- | 27 kDa γ- | − |
| | | | endochitinase | γ-zein | zein; | zein | |
| | | | b | | 3 repeats | | |
| PBT-6 | 19 | 68 | 16 kDa γ- | 27 kDa | Cowpea | 27 kDa γ- | − |
| | | | zein | γ-zein | γ-zein | zein | |
| | | | (ABD63259) | | ortholog | | |
| PBT-8 | 21 | 69 | 27 kDa γ- | 27 kDa | Cowpea | 27 kDa γ- | + |
| | | | zein | γ-zein | γ-zein | zein | |
| | | | | | ortholog | | |
| PBT-9 | 22 | 70 | γ-kafirin | γ-kafirin | γ-kafirin; | γ-kafirin | − |
| | | | | | 2 repeats | | |
| PBT-17 | 30 | 71 | 16 kDa γ- | γ-kafirin | Cowpea | γ-kafirin | + |
| | | | zein | | γ-zein | | |
| | | | (AAL16978) | | ortholog | | |
| PBT-18 | 31 | 72 | 16 kDa γ- | γ-kafirin | Cowpea | γ-kafirin | + |
| | | | zein | | γ-zein | | |
| | | | (ABD63259) | | ortholog | | |
| PBT-19 | 32 | 73 | 50 kDa γ- | γ-kafirin | Cowpea | γ-kafirin | + |
| | | | zein | | γ-zein | | |
| | | | | | ortholog | | |
| PBT-20 | 33 | 74 | 27 kDa γ- | γ-kafirin | Cowpea | γ-kafirin | + |
| | | | zein | | γ-zein | | |
| | | | | | ortholog | | |
| Modified | 63 | 64 | γ-kafirin | NA | γ-kafirin; | γ-kafirin | − |
| PBT-9 | | | | | 2 repeats | | |
| to omit | | | | | | | |
| spacer | | | | | | | |
| Modified | 57 | 58 | 50 kDa γ- | NA | 27 kDa γ- | 27 kDa γ- | − |
| PBT-1 | | | zein | | zein; | zein | |
| to omit | | | | | 1 repeat | | |
| spacer | | | | | | | |
| Modified | 59 | 60 | 50 kDa γ- | NA | 27 kDa γ- | 27 kDa γ- | − |
| PBT-3 | | | zein | | zein; | zein | |
| to omit | | | | | 3 repeats | | |
| spacer | | | | | | | |
| Modified | 61 | 62 | 27 kDa γ- | NA | Cowpea | 27 kDa γ- | − |
| PBT-8 | | | zein | | γ-zein | zein | |
| to omit | | | | | ortholog | | |
| spacer | | | | | | | |
| PBT-21 | 34 | 75 | 50 kDa γ- | 27 kDa | 27 kDa γ- | γ-kafirin | + |
| | | | zein | γ-zein | zein; | | |
| | | | | | 3 repeats | | |
| PBT-22 | 35 | 76 | 16 kDa γ- | 27 kDa | 27 kDa γ- | γ-kafirin | + |
| | | | zein | γ-zein | zein; | | |
| | | | (AAL16978) | | 3 repeats | | |
| PBT-23 | 36 | 77 | 16 kDa γ- | 27 kDa | 27 kDa γ- | γ-kafirin | + |
| | | | zein | γ-zein | zein; | | |
| | | | (ABD63259) | | 3 repeats | | |
| PBT-24 | 45 | 78 | 27 kDa γ- | 27 kDa | 27 kDa γ- | γ-kafirin | + |
| | | | zein | γ-zein | zein; | | |
| | | | | | 3 repeats | | |
| PBT-25 | 46 | 79 | 50 kDa γ- | γ-kafirin | 27 kDa γ- | 27 kDa γ- | + |
| | | | zein | | zein; | zein | |
| | | | | | 3 repeats | | |
| PBT-26 | 47 | 80 | 50 kDa γ- | 27 kDa | γ-kafirin; | γ-kafirin | + |
| | | | zein | γ-zein | 2 repeats | | |
| PBT-27 | 48 | 81 | 16 kDa γ- | 27 kDa | γ-kafirin; | γ-kafirin | + |
| | | | zein | γ-zein | 2 repeats | | |
| | | | (AAL16978) | | | | |
| PBT-28 | 49 | 82 | 16 kDa γ- | 27 kDa | γ-kafirin; | γ-kafirin | + |
| | | | zein | γ-zein | 2 repeats | | |
| | | | (ABD63259) | | | | |
| PBT-29 | 50 | 83 | 27 kDa γ- | 27 kDa | γ-kafirin; | γ-kafirin | + |
| | | | zein | γ-zein | 2 repeats | | |
| PBT-30 | 51 | 84 | 50 kDa γ- | γ-kafirin | γ-kafirin; | 27 kDa γ- | + |
| | | | zein | | 2 repeats | zein | |
| PBT-31 | 52 | 85 | 50 kDa γ- | 27 kDa | Cowpea | γ-kafirin | + |
| | | | zein | γ-zein | γ-zein | | |
| | | | | | ortholog | | |
| PBT-32 | 53 | 86 | 16 kDa γ- | 27 kDa | Cowpea | γ-kafirin | + |
| | | | zein | γ-zein | γ-zein | | |
| | | | (AAL16978) | | ortholog | | |
| PBT-33 | 54 | 87 | 16 kDa γ- | 27 kDa | Cowpea | γ-kafirin | + |
| | | | zein | γ-zein | γ-zein | | |
| | | | (ABD63259) | | ortholog | | |
| PBT-34 | 55 | 88 | 27 kDa γ- | 27 kDa | Cowpea | γ-kafirin | + |
| | | | zein | γ-zein | γ-zein | | |
| | | | | | ortholog | | |

NA: Not applicable
+: Protein bodies were detected using anti-His tag antibodies (Western Blotting) in BMS corn cell culture following the method described in Example 10 in transgenic plant tissues tested.
−: Protein bodies were not detected using Western Blotting anti-His tag antibodies in BMS corn cell culture following the method described in Example 10 in transgenic plant tissues tested.
*The C-terminus of SEQ ID NO: 38 corresponds to positions 112 to 223 of the amino acid sequence of SEQ ID NO: 38.

1. A modified protein body tag comprising a signal peptide domain, a spacer domain, a repeat domain comprising one or more repeat units, and a Pro-X domain,
wherein
(i) at least one repeat unit of the repeat domain is heterologous to the Pro-X domain,
(ii) the signal peptide domain is from a different protein from the same species as the Pro-X domain,
(iii) at least one of the domains but not all of said domains is from a γ-kafirin protein, and/or,
(iv) the spacer domain is heterologous to the repeat domain or the Pro-X domain.

2. The modified protein body tag of claim 1, wherein at least one of the domains is obtained from a γ-zein protein or homolog thereof.

3. The modified protein body tag of claim 2, where the γ-zein protein or homolog thereof is selected from the group consisting of a 27 kDa γ-zein protein, a 50 kDa γ-zein protein, a 16 kDa γ-zein protein, a γ-kafirin, and a cowpea γ-zein ortholog.

4. The modified protein body tag of claim 1, wherein the signal peptide comprises the sequence of SEQ ID NO: 1, SEQ ID NO: 2, SEQ ID NO: 3, SEQ ID NO: 4, or SEQ ID NO: 5; wherein the repeat domain comprises one or more repeat units of the sequence SEQ ID NO: 8, SEQ ID NO: 10, or SEQ ID NO: 11; wherein the Pro-X domain comprises the sequence of SEQ ID NO: 12 or SEQ ID NO: 13; and wherein the spacer domain comprises the sequence of SEQ ID NO: 6 or SEQ ID NO: 7.

5. A modified protein body tag comprising a signal peptide domain, a spacer domain, a repeat domain comprising one or more repeat units, and a Pro-X domain, wherein at least one domain is from a γ-kafirin protein and the repeat domain has a different number of repeats units than a wild-type γ-kafirin repeat domain.

6. A polypeptide comprising the modified protein body tag of claim 1, wherein the modified protein body tag comprises the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55.

7. A nucleic acid molecule comprising a nucleic acid sequence encoding the modified protein body tag of claim 1.

8. The nucleic acid molecule of claim 7, wherein the nucleic acid sequence is codon optimized.

9. An expression cassette comprising the nucleic acid molecule of claim 7, at least one nucleic acid molecule encoding a protein of interest, and at least one regulatory sequence for expressing the at least one nucleic acid molecule in a host cell.

10. The expression cassette of claim 9, wherein the regulatory sequence comprises a promoter selected from the group consisting of seed-specific promoters, constitutive promoters, tissue-specific promoters, ubiquitous promoters, inducible promoters, and developmentally regulated promoters.

11. A vector, plant cell, plant tissue, plant or part thereof comprising
a) the nucleic acid molecule of claim 7; or
b) an expression cassette comprising the nucleic acid molecule, at least one nucleic acid molecule encoding a protein of interest, and at least one regulatory sequence for expressing the at least one nucleic acid molecule in a host cell.

12. The plant cell, plant tissue, plant or part thereof of claim 11, wherein the plant cell, plant tissue, plant or part thereof is obtained from tobacco, Arabidopsis, maize, rice, wheat, barley, soybean, canola, rapeseed, cotton, sugarcane, or alfalfa.

13. The plant cell, plant tissue, plant or part thereof of claim 11, wherein the nucleic acid molecule comprises
a) a nucleotide sequence encoding the amino sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55, or
(b) a nucleotide sequence comprising the nucleotide sequence of (a) codon optimized for maize.

14. A host cell system for evaluating protein body targeting and/or formation and/or accumulation of a protein of interest in a protein body, comprising one or more host cells which comprise
a) a nucleic acid molecule comprising a nucleic acid sequence encoding the modified protein body tag of claim 1; and
b) at least one nucleic acid molecule encoding a protein of interest.

15. A method for evaluating protein body targeting and/or formation and/or accumulation of a protein of interest in a protein body, comprising
a) providing the host cell system of claim 14; and
b) evaluating the protein body formation and/or the expression and/or accumulation of the protein of interest in the host cells of said system.

16. A method for designing a protein body tag of reduced allergenicity, which comprises
a) providing amino acid sequences which encode the signal peptide domain, the spacer domain, repeat domain, and Pro-X domain of a protein body tag, which sequences together define the amino acid sequence of a designed protein body tag;
b) comparing the sequence of said designed protein body tag to a database of allergenic proteins to identify areas of homology, if any, between the designed protein body tag and the proteins contained in the database, which areas of homology signify potential allergenicity; and
c) identifying designed protein body tags having no or few areas of homology which signify potential allergenicity as indicated by said comparison.

17. The method of claim 16, where the areas of potential allergenicity are defined by 8 contiguous amino acids or are defined by 80 contiguous amino acids.

18. The method of claim 16, where the domains of said protein body tag are selected such that at least one domain is heterologous to at least one other domain.

19. A protein body tag identified as a designed protein body tag by the method of claim 16, where the protein body tag has a non-wild-type sequence.

20. The protein body tag of claim 19, which comprises a modified protein body tag comprising a signal peptide domain, a spacer domain, a repeat domain comprising one or more repeat units, and a Pro-X domain,
wherein
(i) at least one repeat unit of the repeat domain is heterologous to the Pro-X domain,
(ii) the signal peptide domain is from a different protein from the same species as the Pro-X domain,
(iii) at least one of the domains but not all of said domains is from a γ-kafirin protein, and/or,
(iv) the spacer domain is heterologous to the repeat domain or the Pro-X domain.

21. The protein body tag of claim 19, wherein the designed protein body tag comprises the sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55.

22. A seed or progeny of the plant of claim 11, which comprises said nucleic acid molecule or expression cassette.

23. A fusion protein comprising
(a) a first polypeptide comprising
(i) the modified protein body tag of claim 1;
(ii) the polypeptide sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55; or
(iii) a modified protein body tag with reduced allergenicity comprising the polypeptide sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55;
and
(b) a second polypeptide comprising at least one protein of interest.

24. A method for protein body targeting and/or formation and/or accumulation of a protein of interest comprising utilizing the protein body tag of claim 1 for protein body targeting and/or formation and/or accumulation of a protein of interest in a plant cell, plant tissue, plant or part thereof.

25. A method for production of a protein of interest comprising
(a) culturing or growing the plant cell, plant tissue, plant or part thereof of claim 11 or transgenic cells, cell cultures, parts, tissues, organs or propagation material derived therefrom under conditions that provide for expression of the protein of interest; and optionally
(b) isolating the protein of interest.

26. A method for the production of a foodstuff, feedstuff, seed, pharmaceutical, or protein of interest comprising
(a) growing or culturing the plant cell, plant tissue, plant or part thereof of claim 11 or transgenic cells, cell cultures, parts, tissues, organs or propagation material derived therefrom; and
(b) producing and/or isolating the desired foodstuff, feedstuff, seed, pharmaceutical, or protein of interest from the plant cell, plant tissue, plant or part thereof or transgenic cells, cell cultures, parts, tissues, organs or propagation material derived therefrom.

27. A method of producing a transgenic plant which targets a protein of interest to a protein body, the method comprising:
a) transforming a plant cell with an expression cassette comprising
i) a first nucleotide sequence comprising a nucleotide sequence encoding the modified protein body tag of claim 1; and
ii) a second nucleotide sequence encoding a protein of interest; and
b) regenerating a transgenic plant from the plant cell.

28. The method of claim 27, wherein the expression cassette comprises at least one other nucleotide sequence encoding a further protein of interest.

29. The method of claim 28, wherein the further protein of interest is overexpressed or down-regulated.

30. The method of claim 28, wherein the further protein of interest comprises an α-zein protein.
